(19) United States
(12) Patent Application Publication
(10) Pub. No.: US 2008/0175836 A1
(43) Pub. Date: Jul. 24, 2008

(54) Immunogenic composition based on conditionally live virion and method for producing the same

(76) Inventor: Nelson M. Karp, Virginia Beach, VA (US)

Correspondence Address: WILLIAMS MULLEN, 222 CENTRAL PARK AVENUE, SUITE 1700, VIRGINIA BEACH, VA, 23462, US

(21) Appl. No.: 11/634,508

(22) Filed: Dec. 6, 2006

Related U.S. Application Data
(60) Provisional application No. 60/749,007, filed on Dec. 9, 2005.

(51) Int. Cl. A61K 39/12; C12N 7/01; A61K 39/395

(52) U.S. Cl. 424/130.1; 435/235.1; 424/199.1

(57) ABSTRACT

A conditionally live virion and method for making the same whereby the viral DNA or RNA is modified so that the virion is incapable of replication unless a protein supplement is added to the expression system. The expression system is either a traditional cell culture or cell free expression system suitable for self assembly of viral particles. Both expression systems require the addition of viral proteins either for replication or assembly of the replication incompetent virion. The conditionally live viron is then used for creating a vaccine with three fold immunogenic properties that are elicited by 1) the whole intact replication incompetent virus; 2) the conditionally live virion temporally resuscitated by addition of protein supplements; and 3) the protein supplement itself acting as a subunit vaccine.

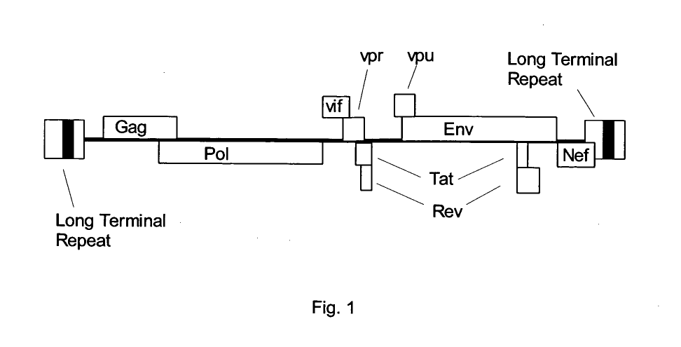

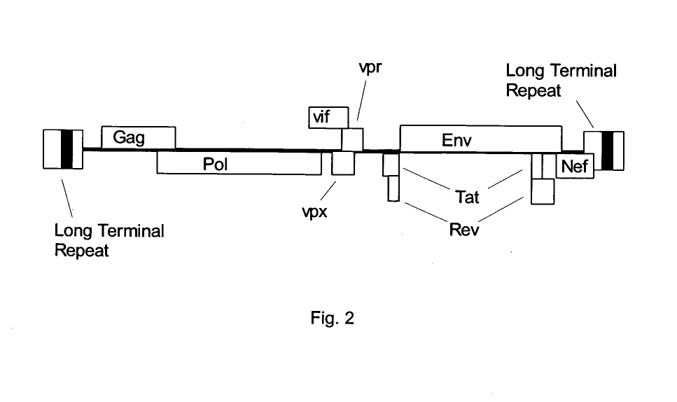

### RELATED U.S. APPLICATION DATA

Provisional application No. 60/749,007, filed on Dec. 9, 2005.

### BACKGROUND OF THE INVENTION

1. field of the Invention

The present invention relates to an immunogenic composition and the method of manufacturing the same.

2. Background

Despite profound efforts, there is no safe, curative vaccine for HIV. Various steps of the HIV life cycle have been targeted by inventors. To date, research has not found a composition that would foster an effective immune response against the immunosuppressive retroviruses HIV-1 and HIV-2. Most HIV vaccines use portions of the envelopes of surface glycoproteins (gp160, gp120, and gp41) of the virus in an attempt to induce production of neutralizing antibodies against the envelope spikes of the virus. Some have been successful in producing high titers of neutralizing antibodies. The thought behind this approach is that the antibodies that bind to these glycoproteins would neutralize the virus and prevent infection. A functioning immune system could then activate the complement system, which would cascade to lysis and destroy the virus. However, the virus is able to evade the immune system with alacrity and ease. To date there has not been a single recorded case study of an individual contracting AIDS, mounting an appropriate immune response and eliminating the virus. Therefore there is no marker for immunity with HIV disease.

A number of drugs or compositions (e.g., AZT, ddI, ddC, d4T and 3TC) inhibit reverse transcription. These 2′,3′-dideoxynucleoside analogs can be effective against certain strains, but are vulnerable to the genomic mutability of HIV. (Deeks, Ch. 6) These medications also face problems of toxicity, cost, complex treatment regimens, drug-drug interactions, as well as drug resistance.

Vaccines to a pathogen are more limited in scope than antimicrobial therapy. An antibiotic may have multiple approved uses for a variety of bacterial infections from different sources. A vaccine, however, if effective only protects the individual from contracting a specific disease. The margins can be blurred, however, if a vaccine consists of a pathogen from a related infectious element. The classic example of this is the smallpox vaccine, which is vaccinia. This virus resembles buffalopox and may have been derived from passage of cowpox and/or smallpox through animal vectors. (Flint, 2004, p. 6) In the 19th century Edward Jenner noted that patients who were exposed to cowpox were immune to smallpox. Following this observation, he developed a cowpox derived vaccine for the prevention of smallpox. A Jennerian-type vaccine utilizes one pathogen to elicit an immune response to a second pathogen. (Wagner, et al., pp. 102-108)

Current available vaccines fall into one of eight broad categories: (1) live attenuated (Sabin Polio, Measles, Mumps, Rubella, Yellow Fever, Varicella Zoster (Chickenpox), BCG (Tuberculosis), Typhoid Fever (Salmonella typhi), Rabies (for dogs and other animals); (2) inactivated whole virus or bacteria Rabies (for humans), Influenza, Hepatitis A, Pertussis (Bordatella pertussis), Paratyphoid fever (Salmonella paratyphi), typhus fever (Rickettsia prowazekii), Plague (Yersinia pestis)); (3) subunit (Hepatitis A or B); (4) inactivated toxin or toxoid (Tetanus, Diphtheria); (5) Jennerian (smallpox); (6) Recombinant Live (Rabies for animals utilizing vaccinia vector); (7) Conjugated (Meningitis), and (8) Purified Capsular Polysaccharide (Meningitis (Haemophilus influenza) and Streptococcal pneumonia.

Live vaccines create an actual infection within the host. Therefore the humoral and cell mediated arms of the immune system respond in a coordinated rhythmical fashion to eradicate the infection. As a result, long term if not lifetime immunity is possible. Another advantage of a live vaccine can be realized if the vector is excreted by the immunized host. An un-immunized patient can contract the infection and consequently become immune. An inadvertent and often deadly consequence of this could occur if an un-immunized patient was not a suitable candidate. Nonetheless the concept of “herd immunity” can best be realized with a live vector. (Levinson, pp. 247-243) Often only one vaccination is required. Live vaccines usually consist of an attenuated, non-virulent, or relatively non-virulent vector. A disadvantage of this vaccination method is the potential for a back mutation to occur rendering the organism virulent. Furthermore, some individuals will succumb to a relatively avirulent vector often due to an underlying immunologic disease, concurrent illness, or a preexisting condition. A classic example of this would be the administration of a smallpox vaccine to a patient with eczema or psoriasis but otherwise in good health. These patients often developed disseminating fulminant disease and succumbed to the vaccine.

Killed whole virus or bacterial derived vaccines are characterized by a large safety margin. An infectious disease will not result from a vaccine if the virus has lost the ability to replicate. Therefore, the effect of “herd immunity” is not applicable to the Salk vaccine in the same way that it is with the Sabin vaccine. A disadvantage of killed whole virus vaccines is that they generally produce a weak immune response, if any. Without pathogen replication, immunologic recognition often does not occur. An additional disadvantage is the lack of a systemic response to a killed or replication incompetent vector. The Salk vaccine for polio, an engineered inactivated vaccine, does not result in mucosal immunization. In other words an IgA response is not realized. Furthermore, a cytotoxic T cell response does not occur or is often ineffective because of the lack of intracellular replication with inactivated vectors. Without intracellular replication, as seen in live vector vaccines, viral proteins do not enter into the cytosolic proteasomal, TAP, endoplasmic reticulum, Golgi pathway necessary for association of the viral epitopes with MHC-I protein. Pathogen epitopes presented in the context of MHC-I proteins elicit CD8⁺ (or Th-1) responses.

Killed vaccines undergo an alternative immunologic response to pathogen epitopes. Internalization of a killed vaccine occurs as a result of either endocytosis or phagocytosis. Whichever uptake mechanism is used, an intracellular organelle known as an endocytic vesicle or phagosome is a result. The membrane of the vesicle is derived from the plasma membrane and the content of the lumen contains cytoplasm and extracellular derived material. Through the action of proton pumps on the vesicle membrane, hydrogen atoms are actively transported into the vesicle, acidifying the contents. These vesicles then fuse with lysosomes that contain a variety of enzymes which are active in an acidic environment. The resulting phagolysosomes degrade the vesicular contents to produce a variety of peptides and glycoproteins. Within this structure, the pathogen derived fragments come in contact with MHC-II proteins. These proteins are synthesized within the endoplasmic reticulum and are transported to the phagolysosomes via the Golgi apparatus. MHC-II proteins interact primarily with CD4+ cells eliciting a Th-2 biased immune response that is limited in scope to the immunologic sphere in which it is encountered. Therefore killed or inactivated vectors elicit primarily a humoral or antibody response and mucosal immunity is not realized unless it is mucosally administered. Additionally, killed vectors usually require multiple administrations of the vaccine and the immunologic memory response noted is often shorter in duration than that seen with a live vector. (Parham, 2005, pp. 67-96; Levinson, pp. 393-412; Kaufmann; 1997, pp. 37-45)

Subunit vaccines direct the immunologic response to a critical structural component of the invading organism. Since no replication occurs, a large safety margin exists. The safety comes at a price: a weak, narrowly defined immunologic response, which is primarily Th-2 biased.

This is in contrast to a Th-1 biased immune response which is preferred for all intracellular replicating pathogens including viruses. T cells respond to antigens only in the context of MHC molecules on antigen presenting cells (B cells, macrophages and dendritic cells). (Peter Parham, 2005, Ch. 3, pp. 67) More specifically, a Th-1 response is dependent upon presentation of antigen bound to a MHC-1 protein. Intracellularly replicating organisms are degraded by the TAP-proteasome pathway. (Peter Parham, 2005, Ch. 3, pp. 67-96) The cell directs proteolysis into this pathway by conjugating the protein with multiple ubiquitin residues through a hierarchical series of enzymes (E1, E2 and E3). (Krauss, pp. 101-113; Parham, 2005, pp. 81)

Nucleotide based vaccines (DNA or RNA) use HIV genes. The host cellular transcription and translational machinery produces the HIV proteins. An immunologic response to these proteins is anticipated. The nucleic acid itself is not the focus of the proposed immunologic response, but does elicit cellular effector mechanisms designed to destroy it, often rendering the vaccine ineffective. Viral nucleic acid is recognized by various components of the innate immune response as “foreign”. Before transcription and translation of the viral nucleic acid commences the host has eliminated it. If properly administered HIV disease does not result. To be potentially effective, nucleotide based vaccines must accomplish several steps. These include but are not limited to the following. The first step is cellular uptake of intact, unmodified viral nucleic acid. The second step is evasion of multiple host cytoplasmic enzymes directed towards destruction of pathogen derived nucleic acid. Many components of the innate immune response are so directed. An innate response to viral nucleic acid does not elicit immunity. Therefore a protective vaccination effect cannot be realized if the viral nucleic acid is destroyed within the cytoplasm or nucleoplasm in the cell. The third step is assimilation of viral nucleic acid into the nucleus. This can be accomplished either by passing through the nuclear pores which is a highly regulated process or bursting through the nuclear membrane which can potentially disrupt normal cellular function and interfere with the proposed immune response. The fourth step is incorporation of viral nucleic acid into the host DNA. The fifth step is transcription of viral gene(s). The sixth step is translation of viral genes in host cytoplasm. All these steps must be accomplished within the proper immunologic sphere before a cellular response can occur. Accomplishment of these steps however, is not to be equated with an effective immunologic response. Nucleotide vaccines are not commercially available. Thus, there is need for an HIV vaccine with a high level of safety and efficacy.

### DESCRIPTION OF THE INVENTION

### Summary of the Invention

The present invention is an immunogenic composition or vaccine, and a method to produce an immunogenic composition having the high safety features of a subunit vaccine combined with the effectiveness of a live vaccine, which is capable of eliciting a Th-1 biased immune response. An alternative to conventional approaches, the present invention is based on a conditionally live virus; that is, an otherwise replication incompetent virus is enabled to be replication competent for a limited time upon the addition of exogenous protein, which substitutes for protein that is unavailable due to a modification or deletion of the corresponding genetic sequence encoding that protein in the viral genome (or “conditionally live”). One embodiment of the present invention uses a knockout virion in which one or more specific viral proteins are targeted. In each embodiment of the present invention, the protein deficit corresponding to the “knocked-out” targeted genetic sequence may be exogenously added. It is contemplated that a predetermined quantity of exogenously added targeted protein may be necessary to enable the otherwise replication incompetent virion to achieve a desired temporally and quantitatively defined or limited level of replication. The quantity, half life, intracellular concentration, intracellular location, and conformational structure of exogenously added protein provided in cocktail with the conditionally live virus will control the replication kinetics of the immunogenic composition or vaccine.

One aspect of the present invention is that the host is exposed to the complete or near complete repertoire of immunogens comprising the pathogen in the context of an infection that may appear normal to the immune system. This approach embraces the efficacy, breadth of immunologic response, and long term memory of a live viral vector. Depending on the context of the administered composition or vaccine, herd immunity may also be realized. In addition, the present invention realizes the safety of a killed or subunit vaccine because the resulting virion will be replication incompetent in the absence of exogenous protein corresponding to the modified replication protein gene or corresponding mRNA.

The present invention may be considered as having two components, each of which is immunogenic. The first component is an intact virion modified in the viral DNA or mRNA to have defective sequences devoid of part or all of the gene(s) or mRNA encoding one or more proteins. Alternatively the genes encoding the targeted proteins may be substituted with non-translatable nucleic acid. The second component is/are the addition of one or more exogenous proteins corresponding to those not encoded in the viral DNA or RNA sequence. The immunogenicity of the first component is biphasic. Administered without the complementing deficient protein(s), an immunogenic composition or vaccine based on a whole viral replication incompetent virion is realized. Administered with the complementing exogenous protein, a conditionally live virion, temporally controlled and limited in replication may be achieved, improving safety. Once replication ceases, a replication incompetent and non-infectious virion remains. Therefore, the starting point and ending point of this composition is the same: a replication incompetent whole virion, which can function as an immunogenic composition.

In one embodiment of the present invention, a Th-1 response will be elicited and directed to one or more components of the intact virion. The administered or added protein(s) within the concept of an intact host immunologic response is a subunit vaccine. Because the first component is biphasic, the immunogenic composition is in reality three vaccine concepts administered simultaneously: (1) a whole intact replication incompetent virus; (2) a (conditionally) live virion (temporally controlled with a half life measured in hours or days); and (3) a subunit vaccine(s). Conceptually, each component of the vaccine will serve as an adjuvant for the other two, enhancing the overall immunogenicity of the vaccine formulation. Multiple vector vaccines such as the MMR and DPT have demonstrated positive responses to each component. Each vector of the trivalent vaccines enhances the immunogenicity of the other two.

One embodiment of the present invention provides a rapid system for creating a conditionally live vaccine in combination without tissue culture. Tissue culture derived antigens are compromised by the assimilation of tissue derived antigens to which an unanticipated immune response could occur limiting the effectiveness of the vaccine. Furthermore, the virus in tissue culture will continue to mutate in response to the host cell environment. A tissue culture does not accurately recreate the intact host immune systems. Virus derived from tissue culture will not mirror field or clinical isolates. Continued viral mutation in tissue culture is unpredictable leading to a lack of reproducibility, compromising quality control in virion replication and harvesting for vaccine production. Therefore tissue derived virus is suboptimal for vaccine administration.

The present invention does not rely on a live recombinant carrier. Initial immune responses to recombinant vectors are restricted in part or in whole to the carrier vector itself compromising efficacy. Subsequent vaccine challenges of a live recombinant carrier elicit a quick adaptive immune response to the carrier vector eliminating the vector and genetically engineered antigenic material. The “original antigenic sin” concept does not allow for a broader immune response with subsequent vaccine challenges. Repeat vaccine challenges enhance the acquired immune response in terms of specificity and robustness but the response is only to those antigens to which an immunologic response was initially directed. Therefore recombinant vectors are antigenically limiting in immune recognition and are limited to one application. In other words, a recombinant vector system allows for single use per individual since after the first exposure, the individual will develop immunity to the vector itself. Additionally, recombinant vector systems present to the immune system a wide variety of antigenic material to which an immunologic response is not desired. The vector itself is the source of this antigenic decoy. The potential numbers of antigens comprising the carrier vector itself greatly exceed that of the genetically introduced material to which an immunologic response is desired. Furthermore, the exterior proteins of the recombinant vector are the first antigens to which the immune system is exposed. “Booster doses” of recombinant vectors are not beneficial. This invention circumvents these disadvantages currently faced by vaccines introduced in recombinant vector systems.

### BRIEF DESCRIPTION OF THE FIGURES

A schematic view of the linear genome of HIV-1 with coding sequences of the HIV genes depicted as open rectangles.

A schematic view of the linear genome of HIV-2 with coding sequences of the HIV genes depicted as open rectangles.

### DETAILED DESCRIPTION

### Introduction

The present invention is based on a conditionally live virion; that is, a virion modified to be otherwise replication incompetent is enabled to be replication competent for a limited time upon the addition of exogenous protein, which substitutes for protein that is unavailable due to the modification (or deletion) of the corresponding genetic sequence encoding that protein in the viral genome. A virus by definition is not a live or dead structure. It is best characterized as being replication competent or replication incompetent. In this invention, a live virus refers to a replication competent vector. One aspect of the present invention is an immunogenic composition comprising a viral DNA or RNA representing a complete viral genome in which at least one replication protein gene or corresponding mRNA has been modified to render the viral DNA or RNA replication incompetent; this modified viral DNA or RNA is then encapsulated by viral proteins that self assemble in a cell free expression system, forming a conditionally live virion. The method for producing this conditionally live virion includes the steps of providing at least one viral DNA or RNA molecule representing a complete genome, amplifying the viral DNA or RNA, modifying the viral DNA or RNA in at least one replication protein gene or corresponding mRNA, collecting the amplified and modified viral DNA or RNA, repackaging the collected DNA or RNA in a cell free expression system suitable for self assembly of viral particles, and collecting a desired quantity of the resulting conditionally live virions. An alternative method for producing this conditionally live is using a traditional cell culture system. In this method, a virion modified in at least one replication protein gene or corresponding mRNA may be cultured under conditions suitable for viral replication with the addition of exogenous protein corresponding to the at least one replication protein gene or corresponding mRNA. Therefore, a fourth aspect of the present invention is formulating a vaccine using the replication incompetent virion in combination with whole viral proteins, protein fragments, protein derivatives, or combinations thereof. A vaccine created by either method will have three fold immunogenic properties that are elicited by 1) the whole intact replication incompetent virus; 2) the conditionally live virion temporally resuscitated by addition of protein supplements; and 3) the protein supplement itself acting as a subunit vaccine. An added feature of a vaccine formulated with the conditionally live virion created in the cell free system is that no vector is present to contribute to the elicited immunogenic response of the vaccine when administered.

### Preferred Targeted Nucleotide Sequence Replication Protein Gene or Corresponding mRNA

Preferably, for an embodiment directed to HIV, targeted nucleotide sequence(s) are located within the central region of the HIV genome and are necessary for viral replication. Other nucleic acid sequence(s) according to the present invention may be targeted for deletion or substituted with non-translatable genetic information as well. These include but are not limited to the envelope glycoproteins, gp120 and gp41, the retroviral encoded enzymes (protease, reverse transcriptase, integrase and RNAaseH), Nef and the long terminal repeat sequences so long as the overall modification results in a replication incompetent virion. In general, however, for the purposes of this application, a replication protein gene is a gene that may be modified or deleted to render the virion replication incompetent when in the intact host. Thus, replication protein gene or corresponding mRNA for the purpose of this application means the nucleic acid sequence encoding the protein. However, the protein(s) missing in the transcription of the viral genome can be exogenously added. This will result in active “normal” viral replication in an intact host. Also for the purposes of this invention, modifying (or modification of) a replication protein gene should be construed broadly, so as to include deletion or mutation, for example, so long as the virion would be rendered replication incompetent in the absence of exogenous replication protein, as described further herein. In one HIV embodiment of the present invention, for example, the targeted viral proteins are Vif, Vpr, Vpu (HIV-1), Tat exon 1, and Vpx (HIV-2). Relatively small in size, they are also encoded, in part, by non-overlapping segments and are all essential proteins for viral replication. Removal of one or more of these non-overlapping genomic segments will yield a virus incapable of in vivo reproduction unless an exogenous source of the defective or deficient protein is supplied.

The vif nucleotide sequence is located 3′ to the pol nucleotide sequence and 5′ to the vpr nucleotide sequence. In some viral isolates a small overlap exist between the nucleotide sequences at the 3′ terminus of pol and the 5′ terminus of vif. The 3′ terminal nucleotide sequence of vif overlaps with the 5′ terminus of vpr. The vif protein is encoded by one exon. A non-overlapping segment of vif between pol and vpr can be selectively excised rendering the virus vif defective without adversely affecting the transcriptional and translational products of pol and vpr.

The Vif protein (Viral infectivity factor) is incorporated into both HIV-1 and HIV-2 virions through an interaction with the viral RNA and nucleoprotein complexes. Vif is approximately 216 amino acids long. Vif is not a structural protein and is Rev dependent and therefore produced late in the viral life cycle. Vif defective virions in vitro are 103 times less infectious than the intact virus with a functional vif gene. Vif defective virions in vivo are replication incompetent. Vif has multiple functions including but not limited to the following:
1. Increases viral infectivity.
2. Enhances virion assembly.
3. Promotes viral DNA synthesis by the reverse transcriptase enzyme.
4. Antagonizes cellular protein CEM15/APOBEC3G (apolipoprotein B RNA-editing enzyme or apolipoprotein B RNA-catalytic enzyme). APOBEC3G, a component of the innate immune system, is a cytidine deaminase.
5. Contains an inhibitory sequence (INS) that prevents the premature nuclear export of viral RNA into the cytoplasm.
6. Induces structural changes of the plasma membrane.
7. Contributes to cytokine disregulation, inhibits phagocytosis and limits cell spreading.
8. Protects viral RNA from intracellular RNAse degradation.
9. Temporally regulates activity of the protease enzyme.

A conditionally live virus in which the non-overlapping nucleotide sequence for vif has been spliced out is not capable of viral replication and infection. The Vif protein is produced in excess of that needed within the infected cell. Much of the excess Vif protein is assimilated into non-infected cells where it exerts much if not most of its cytokine dis-regulation and therefore immunosuppressive effect. The exogenous supply of Vif protein not only limits intracellular replication, but also limits vif immunosuppression.

The vpr nucleotide sequence is located 3′ to the vif nucleotide sequence and 5′ to the Tat exon 1 nucleotide sequence. In most viral isolates the 5′ terminus of vpr overlaps with vif and the 3′ terminus overlaps with tat exon 1. Between the 5′ and 3′ overlapping segments is a non-overlapping segment that can be selectively excised. A vpr defective mutant is not capable of active replication in an intact host. The vif and tat exon 1 nucleotide sequences are left intact with selective excision of the intervening non-overlapping segment.

The Vpr protein (viral protein r) is a late gene product of both HIV-1 and HIV-2. Vpr is rev dependent. Vpr is incorporated into virions through interaction with the viral protein p6, which is cleaved from the larger Gag polypeptide.

Vpr has multiple functions including but not limited to the following:
1. Nuclear localizing signal.
2. Coordinates HIV genomic expression with the Tat protein.
3. Blocks cell division of infected T cells in the G₂ phase of the cell cycle.
4. Blocks the cell cycle in the G₁ phase of uninfected T cells.
5. Soluble Vpr arrests non-infected cytotoxic CD8 T cells specific for HIV antigens in the G₂ phase of the cell cycle.
6. Limits B cell somatic hyper mutation necessary for antibody receptor affinity maturation.
7. Enhances viral population heterogeneity by increasing viral mutation.
8. Enhances the activity of p300, a co-activator with histone acetylase activity that regulates gene transcription.
9. Activates transcription factors NF-IL-6 and NF-kB.
10. Interacts with and co-activates the intracellular glucocorticoid receptor.
11. Interacts with and controls the expression of a variety of other cellular proteins including but not limited to Sp1, p53, Rb (hyperphosphorylation), TFIIB, the nuclear transport factors importin-α and nucleoporin Pom21 and the human homologue of MOV34. A cooperative interaction of the Vpr protein and the cellular proteins, p53 and Sp1 has a positive effect on HIV-1 gene transcription.
12. Induces cellular cytoskeletal changes.
13. Induces nuclear membrane herniations possibly contributing to nuclear localization.
14. Induces mitochondrial membrane permeability dysfunction.
15. In HIV-2 Vpr facilitates the incorporation of Vpx into the virions. Vpx is an accessory protein found only in HIV-2 necessary for full viral activity.

A conditionally live virion in which the non-overlapping segment of the vpr nucleotide sequence has been removed is not capable of active replication and infection in an intact host. In an HIV infected cell, the Vpr protein is produced in a quantity in excess of that needed for active replication. The excess Vpr protein is assimilated into uninfected immune cells and functions in part to suppress cell function. In a vpr defective conditionally live virus vaccine, the exogenous supply of Vpr protein limits both viral replication and viral induced immune suppression.

The vpu nucleotide sequence is located between the 3′ terminus of the tat exon 1 nucleotide sequence and 5′ terminus of the env nucleotide sequence. In most viral isolates there is no overlapping nucleotide sequence of tat and vpu. In contrast an overlapping segment of vpu and env is found in most viral strains. The non-overlapping segment of vpu can be selectively excised rendering the virus vpu defective without adversely affecting the transcriptional and translational products of env.

The Vpu protein is only found in HIV-1 and four strains of SIV. The primary amino acid sequence of Vpu is the smallest of the proteins encoded by the HIV genome and varies in length from 77 to 86 amino acids. It is predominantly located within the RER/Golgi apparatus. The Vpu protein is not incorporated into mature virions. The functional equivalent, at least in part, of the Vpu protein in HIV-2 has been ascribed to the Env glycoprotein. Vpu protein production is Rev dependent and therefore is noted late in the viral replication cycle. The messenger RNA encoding the Vpu protein is bicistronic. In most viral isolates the Vpu initiation codon is not in a proper Kozak sequence, or encodes an amino acid other than methionine. For many if not most viral isolates, the initiation sequence of the Vpu protein mRNA is A/GCCAATGG. (The Kozak sequence most easily recognized by the host ribosomal machinery is 5′-ACCAUGG-3′). The initiation codon of the Env glycoprotein is a methionine residue in the proper Kozak sequence. Leaky scanning allows the virus to direct the host transcription machinery to produce the appropriate ratio (1/10) of Vpu/Env proteins.

Although HIV-2 does not encode a Vpu protein, Vpu like activity is found within the gp36 TM subunit. If selection and partitioning of function is a correlate of evolution and a marker of maturation, then HIV-2 is less differentiated than HIV-1. In the phylogenic tree, the more sophisticated organisms develop later in the evolution of life and are better adapted. In evolutionary terms HIV-2 is the “older” virus. Therefore HIV-2 should be an easier target than HIV-1. Indeed HIV-2 has been found to be less infectious and less virulent than HIV-1. Thus, the Vpu protein may be a genetic marker tracking the HIV through time.

The Vpu protein (Viral Protein U) has multiple functions including but not limited to the following:
1. Temporally controls HIV replication.
2. Immunosuppression.
3. Vpu expression enhances viral expression, mutation and dissemination in the host.
4. Decrease host cellular CD4 expression.
5. Contributes to cytokine disregulation and immunologic dysfunction.
6. Facilitates malignant transformation in HIV.
7. Enhances virion release.
8. Decrease MHC-I cellular expression.

A conditionally live virion with an excision of the non-overlapping segment of the vpu nucleotide sequence is incapable of active replication and infection in an intact host. Production of Vpu protein in an HIV infected cell exceeds the cells demand for viral replication. Much of the excess Vpu protein is assimilated into other non-infected immunologic cells. The immunosuppression attributed to the Vpu protein primarily refers to the non-infected cells that have acquired the Vpu protein. In a vpu deficient conditionally live viral vector, exogenous Vpu protein is controlling factor in viral replication. Additionally, the immunosuppression of the Vpu protein will likewise be controlled by the amount and half life of the Vpu protein supplied.

The nucleotide sequence of vpx is 3′ to the nucleotide sequence of vif and 5′ to the nucleotide sequence of vpr. An overlapping genomic sequence between the nucleotide sequences of vif and vpx is found in most HIV-2 strains. In contrast the vpx and vpr nucleotide sequences in most isolates are non-overlapping. Excision of the non-overlapping nucleotide sequence of vpx renders the virus vpx defective without adversely affecting the transcriptional and translational product of the Vif and Vpr proteins.

The Vpx protein (Viral protein x) is packaged into virions by interacting with the p6 domain of Gag. Vpx is found only in HIV-2 and four strains of SIV. Incorporation of Vpx into an intact virion is mediated by a dileucine motif in the N-terminal domain of p6. Approximately equimolar amounts of Gag and Vpx are incorporated into HIV-2 virions. Vpx is a small hydrophobic protein approximately 100 amino acids long with three amphipathic α-helices. Vpx is structurally related to the Vpr protein but functionally different. Vpx lacks a nuclear export signal and is not implicated in cell cycle arrest.

Vpx has multiple functions including but not limited to the following:
1. Nuclear localization signal (NLS).
2. Vpx facilitates HIV-2 infection in non-dividing cells.
3. Facilitates HIV replication in macrophages.
4. Facilitates virion assembly on the cytoplasmic side of the plasma membrane.
5. Interferes with MHC-II antigen presentation.
6. Enhances reverse transcriptase activity.

A conditionally live virion deficient in the nucleotide sequence for vpx is non-viable in an intact host. A cell infected with HIV-2 produces Vpx in excess of its needs. Much of the excess Vpx protein is assimilated into non-infected immune cells. The immunosuppression associated with Vpx occurs in large part in non-infected cells that have incorporated the Vpx protein into the cytoplasm. The exogenous supply of Vpx protein in such a conditionally live virion composition enables not only limited intracellular replication of the virus in infected cells, but also limited immunosuppression exerted by Vpx in non-infected cells.

The tat (Transactivator of Transcription factor) exon 1 nucleotide sequence is located 3′ to the vpr nucleotide sequence and 5′ prime to the vpu nucleotide sequence. An overlapping segment between vpr and tat exon 1 is noted in most viral isolates. A non-overlapping segment encompasses the rest of the tat exon 1 nucleotide sequence. In most isolates that tat exon 1 nucleotide sequence does not overlap vpu in HIV-1. Vpu nucleotide sequence is not incorporated in the HIV-2 genome. A tat defective virion (exon 1) is replication incompetent in an intact host.

The complete HIV-1 Tat protein is encoded by two separate exons. Through alternative splicing, two forms of Tat protein are produced in HIV infected cells. The first 72 amino acids (NH₂ domain) of the Tat protein are essential for viral replication and are encoded by one exon transcript. The second exon encodes the COOH terminal domain encompassing amino acids 73-101. Therefore one form of Tat protein reflects the nucleotide sequence of just one exon encoding the NH₂ domain and is 72 amino acids long. The other form is a product of both exons and is 101 amino acids long (one strain of HIV disease has an 86 amino acid Tat protein). The COOH terminal domain is necessary for the Tat protein to exert many immune modulating affects. Therefore an alternative vaccine may encode only the amino terminal exon of the Tat protein encoded by the Tat exon 1 nucleotide sequence.

The Tat protein is expressed early in the viral replication cycle and is rev independent. The Tat protein is not incorporated into the intact virion.

The Tat protein has numerous functions including, but not limited to the following:
1. Induces NF-κB activation.
2. Inhibit cellular (host), but not viral, mRNA translation.
3. Depletes intracellular cyclin T in both infected and uninfected T cells
4. Down-regulates bcl-2 and induces apoptosis in non infected hematopoietic cells.
5. Up-regulates bcl-2 in HIV infected macrophages interrupting the apoptosis.
6. Induces neuronal death in the central and peripheral nervous systems.
7. Decreases the ability of accessory cells to organize T cell clusters.
8. Activated B cells and induces B cell lymphoma.
9. Induces immunoglobulin synthesis by stimulation of IL-6 release.
10. Inhibits CD26 or dipeptidylaminopeptidase IV activity on T cell membranes blocking recall activation of T cells.
11. Blocks phagolysosomal fusion in monocytes.
12. Inhibits IL-2 and IL2R expression in CD4 cells.
13. Amplifies inflammatory redox state (oxidative stress).
14. Amplifies activity of tumor necrosis factor (TNF)
15. Stimulates TGF-beta release (additional immunosuppression).
16. Represses transcription of MHC I genes.
17. Activates JNK and ERK/MAPK pathways in non-infected CD4 cells.
18. Stimulates monocyte chemotaxis.
19. Represses beta 2-microglobulin promotor.
20. Inhibits IL-12 synthesis.
21. Induces HIV-1 co-receptor synthesis (CCR5 and CXCR4) in non-infected but Tat transfected cells enhancing the susceptibility of uninfected macrophages and T cells to the HIV virus (promotes infectivity of both macrophage and T cell tropic viral strains.
22. Hyperactivates T cells via the CD28 pathway.
23. Enhances growth of Kaposi sarcoma.
24. Inhibits proliferation of uninfected lymphocytes in response to specific antigens.
25. Protects HIV infected T cells from activation induced apoptosis.
26. Induces apoptosis in uninfected T cells.
27. Inhibits Natural Killer (NK) cell cytotoxicity.
28. Up regulates TRAIL production in macrophages.
29. Increases expression of TRAIL in uninfected monocytes.
30. Protects HIV infected monocytes from TRAIL mediated apoptosis.
31. Up-regulates IL-4 receptors on B cells.
32. Induces HIV dementia.
33. Impairment of Dendritic cell function.
34. Reduces mannose receptors on infected and uninfected cells.
35. Enhances transcription of the HIV virus at least one-thousand-fold through protein binding to the transactivation response element (TAR) at the 5′ terminus of HIV mRNAs; specifically interacts with a bulge region in the stem of the TAR element.
36. Augments the activity of the cellular derived RNA polymerase II complex in viral transcription.

Specific examples of an immunogenic composition based on a conditionally live virion and method for producing the same are now set forth below using the Tat protein. However, it will be apparent to one of ordinary skill in the art that many modifications or alternative embodiments are possible, and that specific examples are provided for purposes of illustration only and are not limiting of the invention unless so specified.

For example, a conditionally live virion in which the non-overlapping nucleotide sequence of tat exon 1 is excised is incapable of viral replication and infection. One aspect of the following embodiment is not only limited replication of the conditionally live virion, but also limited immunosuppressive function of the Tat protein as an immunogen and in viral transactivation. The Tat protein is highly conserved among HIV strains. Further, the Tat protein is highly immunosuppressive, and its diverse effects have been document. A cell infected with the HIV virus and actively replicating produces many viral components that are not assimilated into the intact virion or used for viral replication. The Tat protein in such cells is produced in excess of what is needed for replication. The function of excess Tat protein is to suppress the immune system of the host. An exogenous supply of Tat protein for a tat defective conditionally live virion would enable limited replication of the HIV virus and limited Tat mediated immunosuppression. (See, e.g., Rubartelli, et al.)

The HIV Tat protein can be subdivided into several different regions each possessing specific physical, steric and electrostatic properties. A short twenty amino acid sequence consisting of the “core” domain of Tat, specifically amino acids 21-40 is sufficient to propagate HIV in vitro. The Tat protein is encoded in HIV by two separate exons. Therefore, a whole intact replication incompetent virus can be attained through splicing. For example, the first exon may be altered or removed, the second exon may be altered or removed, or both exons may be altered or removed. The first 72 amino acids (NH2 domain) of the Tat protein are essential for viral replication of HIV and are encoded by one exon transcript. The second exon encodes the COOH terminal domain encompassing amino acids 73-101. Therefore, an embodiment of the present invention may be based on the nucleotide sequence of HIV having just one exon encoding the NH₂ domain, and is 72 amino acids long. The COOH terminal domain is necessary for the Tat protein to exert many immune modulating effects. Therefore, another aspect of the present invention may include the nucleotide sequence of HIV encoding only the carboxyl terminal exon of the Tat protein. A further aspect of the present invention may involve splicing mutated nucleotide sequence at one or both exons. The second exon of the Tat protein overlaps into the env gene in totality. The nucleotide sequence of rev exon 2 is completely included within the tat exon 2 nucleotide sequences. To preserve function of the env and rev exon 2 genes special consideration needs to be given. In one embodiment, the splicing sites (either the 5′, 3′ or both the 3′ and 5′ splicing sites can be rendered non-functional terminating tat exon 2 transcription) for the tat exon 2 nucleotide sequence can be mutated in such a manner that splicing at these sites is impossible, but no significant change if any in the amino acid sequence of the env or rev exon 2 gene occurs.

Alternatively, certain specific missense or nonsense nucleotide sequences for tat render the virus replication incompetent. These sequences encoded into an otherwise intact HIV RNA sequence can be used within an intact viral structure. A substitution of glycine for the cysteine residue at amino acid position number 22 (C22G) or 30 (C30G) of the Tat protein abrogates Tat mediated transactivation of the LTR of HIV. Substitution of cysteine residue number 31 with a glycine impairs, but does not totally inhibit, HIV Tat viral transactivation. This would be particularly attractive in a virion encoding only exon I with the above cysteine substitution (C31G), in that viral replication would proceed intracellularly, albeit at a slower pace. Without exon II, most of the immunosuppressive effect of the Tat protein would be missing. (Wang, et al.)

Tat-deficient virions can be obtained by any of a variety of methods. As discussed generally in U.S. Pat. No. 7,132,271 to Lau, which is incorporated by reference. Techniques for producing stable Tat-deficient mutants may include, but are not limited to, with references incorporated: random or site-directed mutagenesis (e.g., Deng, et al.; Busby, et al.), targeted gene deletion (“gene knock-out”) (e.g., Camper, et al.; Aguzzi, et al.), transfection with tat antisense polynucleotides (e.g., Lee et al.) and transfection with a tat dominant negative mutant gene. Thus, Tat mediated immunologic responses may be eliminated by deletion and/or mutation of the nucleotide sequence(s) encoding a bioactive Tat protein without changing the structure of the intact virion since the Tat protein is not included in the intact virion.

In immunogenic compositions completely lacking the non-overlapping nucleotide sequence for the Tat protein encoded by tat exon 1, or encoding a mutated, truncated, or otherwise ineffective Tat protein, a predetermined quantity Tat protein may be added or administered exogenously along with the vaccine itself. This will allow intracellular viral replication for a desired period of time (e.g., hours) until the exogenous Tat protein is exhausted by viral replication, disseminated into the extracellular milieu, or degraded by cellular enzymes. The exogenous Tat protein would need to be in its native non-oxidized form to maintain its ability to transactivate the virus. The Tat protein supplied could embrace one of several forms, which could be used independently, concurrently or sequentially:
1. The complete 101 amino acid sequence;
2. The shorter but still effective 86 amino acid sequence;
3. The truncated NH₂ 72 amino acid sequence encoded by exon I;
4. Other truncated amino acid sequences encoded by exon I possessing transactivating capability as described above with the core domain of Tat protein;
5. A mutated sequence of number 1, 2, 3 or 4 above demonstrating replication competence; or
6. Combination of the above not limited in relative or absolute concentrations or time frame of application.
7. Messenger RNA encoding Tat protein or transcriptionally biologically active fragment.

By including a limited quantity of exogenous Tat protein along with a conditionally live virion (i.e., that lacks the ability to produce the Tat protein itself), the added Tat protein acts as a subunit vaccine that controls viral replication. Immunologic response to the Tat protein, both humoral and cell mediated, has been noted in HIV patients and is inversely correlated with disease progression. By analogy to other multivalent vaccines, such as DPT, the pertussis component performs the function of an adjuvant for the diphtheria and tetanus components, probably by enhancing a local non-specific inflammation. Likewise, the conditionally live virion may act as an immune stimulant for the exogenous Tat protein in the form of a subunit vaccine or vice versa.

Once the limited quantity of exogenous, added Tat protein is exhausted, an inactivated intracellular and extracellular HIV replication incompetent virion remains. This virion possesses the structural components of an infectious, replication competent HIV virion. The missing Tat protein is a regulatory protein involved in viral replication and immunologic suppression; the Tat protein is not a component of the HIV virus. Thus, the present invention achieves intracellular replication of an ultimately replication incompetent virus.

In summary, this example of the present invention is an immunogenic composition in which part or all nucleotide sequencing encoding the Tat protein has been modified (i.e., including deletion or specific mutation). Depending on the application, this may include either or both of the exons encoding the Tat protein. The Tat protein is included within the sphere of the vaccination regimen to allow intracellular HIV replication to proceed. This replication will be short lived and will terminate upon exhaustion of the Tat protein. This embodiment of the present invention described above is exemplary only, and not intended to be limiting.

### Selection of Source Material and Strain(s) of HIV Virus

Classically, a vaccine for one pathogen is comprised of one, two, or possibly three separate but related vectors. For example the Salk and Sabin vaccines are trivalent. This approach would not apply to diseases such as HIV, with its characteristic population demographics (quasi-species) and the plethora of documented strains and circulating recombinant forms of the virus. Formulating a vaccine with an immunogenic composition, generally, is well-known in the art.

The two arms of the present invention may be prepared separately. The following is an aspect of the invention for producing the replication incompetent virion, which encompasses several steps: (1) provision or selection of viral DNA or RNA molecules representing a complete viral genome for the viral strain(s) of interest; (2) isolation of viral nucleic acids, if necessary; (3) nucleic acid modification; (4) nucleic acid amplification; (5) assembly of the replication incompetent whole virion, that is, repackaging the collected nucleic acid in an expression system suitable for self assembly of viral particles; (6) collecting self-assembled conditionally live virions; and (7) optionally adding exogenously replication protein(s) corresponding to the modified gene(s) or corresponding mRNA.

HIV live vectors may be purchased and used as sources of vaccine material from the NIH. However, these viral isolates lack many of the characteristics noted in actively infected patients because they have been passed through numerous cell lines in vitro. Quite typically, continuous cells lines (i.e., cells which have no finite end to the number of mitotic divisions possible) are used as a culture medium due to their universal availability, low cost, well defined nutrient needs and overall predictability. The predictability of continuous cell cultures is defined in three parameters: (1) infinite number of mitosis; (2) short G1 phase of the cell cycle allowing cell division within hours or even minutes; and (3) continual mutation. The virus however, quickly adapts to the host environment. Continuous human T cell lines such as SupT1, H9, Jurkat or A3.01 can also be obtained from the NIH AIDS Research and Reference Reagent Program or the American Type Culture Collection, both in Rockville, Md. Laboratory. Adapted HIV viruses can propagate in these continuous cell lines but most viral isolates of human origin do not. (Michael, et al).

Classical virology distinguishes between “wild-type” virus and mutated or otherwise altered viral material. In actuality, a “wild-type” virus may not be, and often is not, synonymous with virus isolated from an intact host. Therefore a distinction needs to be made between laboratory derived “wild-type,” usually produced by passage through continuous cell cultures and viral isolates from the intact natural host. The latter are best referred to as field or clinical isolates and demonstrate the structural or genetic qualities sought in a vaccine. Thus, virus drawn as a field or clinical isolate from an intact host contrasts with virus from cell cultures.

Within an intact host, the HIV virus inhabits multiple spheres, organ systems, and/or histological tissues, and is excreted in various cellular fluids. The actual HIV virus as well as intact RNA and DNA sequences can be recovered from infected patients at all stages of the disease spectrum, even before the acute retroviral syndrome (i.e., which occurs in most patients within 30 days of infection). Specifically, the virus adapts to its host environment and, with a half life of six hours, a typical HIV virus is produced and secreted by cells in the same tissue that it ultimately re-infects. Therefore, viral cultures in different organ systems of the same patient often demonstrate subtle but important genotypic and phenotypic differences, which are necessary for viral replication in the tissue it infects.

This is an extrapolation on basic Darwinian principles that an organism will adapt to its environment or perish. The immunological milieu of the human host is divided into several separate biospheres or compartments (all of which become HIV infected) including, but not limited to, the gut associated lymphoid tissue (GALT), bronchial associated lymphoid tissue (BALT), skin associated lymphoid tissue (SALT), mammary associated lymphoid tissue (MALT) and conjunctival associated lymphoid tissue (CALT). The lymphocytes and other cellular components, as well as other molecular components, of the immune system are not evenly distributed throughout the somatic tissues. (Parrish, et al.) The immune pressure on the HIV virus therefore differs with its specific tissue or organ of origin. The genotypic and phenotypic expression of the virus will reflect the immune environment it propagates in.

The primary method of HIV transmission is sexual. Therefore the seminal, vaginal, and rectal fluids of intact hosts are logical sources for viral field or clinical isolates for vaccine production. Methods of specimen collection by cervicovaginal lavage are well defined. Manual collection of cervical secretions has also been delineated. This is an alternative method of obtaining either whole replication competent virions, viral RNA or DNA. Viral isolation from seminal fluid is also routinely performed. (Michael, et al., 1999, Ch. 17) Methods of culturing HIV-1 in human semen are standard in the industry. (Michael, et al., 1999, Ch. 8) Finally, the process of collection and processing of rectal secretions has been defined in the literature. (Michael, et al., 1999, Ch. 35)

Detection, isolation, and expansion of the HIV virus can be performed on a variety of infected tissues including, but not limited to, human monocytes/macrophages, T cells, and central nervous system tissue. (Michael, et al., 1999, Ch. 9 and 10) HIV culture and expansion can be accomplished with mitogen-stimulated peripheral blood mononuclear cells (PBMCs) from “normal” uninfected healthy donors. (Michael, et al., 1999, Ch. 1) This process, although the cornerstone of many HIV vaccine and drug efforts, is perilous. The virus will continue to mutate in cell culture and will quickly assume genotypic and phenotypic characterizations (genetic drift) that differentiate it from the original tissue isolate. Cultures may also be unreliable, often requiring 30 days before viral replication is detectible.

Starting materials for isolation of viral nucleic acids can be divided into two broad categories: (1) cell rich; and (2) cell poor. Some overlap in these categories exists. A cell poor isolate can be obtained from an initial cell rich culture. Cell rich starting materials include, but are not limited to, the following: (1) whole blood or blood fractions; (2) bone marrow; (3) tissue specimens, fresh, frozen, paraffin embedded or otherwise prepared; (4) in vitro cultured cells (5) swabs impregnated with tissue derived fluids and cells; and (6) bronchial lavage. Cell poor starting materials include but are not limited to the following: (1) blood plasma; (2) blood serum; (3) urine; (4) saliva (5) cell culture supernatants; and (6) stool. (Botho Bowien, et al.)

Viruses, including HIV, may be isolated from any category of startup materials. However, isolation of viral DNA from cell rich materials will be complicated by the co-purification of host and viral DNA. PCR based technology, as discussed below, can detect, isolate, and amplify viral nucleic acid from cell rich cultures, but this requires a large amount of nucleic acid as template, and this requirement may inhibit PCR. (Bowien, et al., Ch. 5) Viral DNA/RNA in cell rich medium is both cell associated and cell free. In the intracellular compartment, viral nucleic acids may be integrated into the host genome or bound to host and/or viral proteins in both the cytoplasmic and nuclear compartments. Finally, viral nucleic acids in part or in whole can be found in a cell rich system in the extracellular milieu protein free. Therefore in a cell rich medium, the source and content of viral nucleic acid DNA is not uniform.

Cell free body fluids limit, but do not completely eliminate, host DNA contaminants. Viral DNA content in many cell poor isolates is characteristically of low titer, necessitating concentration of nucleic acids before isolation and amplification.

Erythrocytes from mammals are enucleated shortly after entering the circulation, and therefore have very little DNA. Mitochondrial DNA is still found within the mitochondria, but in an intact cell containing a nucleus, the mitochondrial DNA is a very small fraction of the total cellular DNA. Human blood contains approximately 1000 times more erythrocytes than leukocytes which have nuclei. Therefore, if blood is used as a selective medium for viral isolation and amplification, the erythrocytes should be removed first.

This can be accomplished by hypotonic shock, since red blood cells burst more rapidly in a hypotonic medium than white blood cells. Alternatively, Ficoll-density-gradient centrifugation can separate mononuclear cells (lymphocytes and monocytes) from erythrocytes. A third method consists of centrifuging whole blood at 3300 g for ten minutes at room temperature. This separates the blood into three readily discernable fractions: (1) white blood cell enriched fraction known as the buffy coat; (2) blood plasma; and (3) red blood cells. (Bowien, et al., Ch. 2) The buffy coat would be a cell rich source suitable for viral nucleic acid separation, and the blood plasma fraction would serve as a cell poor medium also suitable for viral nucleic acid separation.

Selection of viral strains logically parallels those strains indigenous in the population. As mentioned above, a single clone of virus would not be representative of the HIV epidemic. Other factors to be considered include but are not limited to the immunogenicity and pathogenicity of individual HIV strains. An optimum vaccine should preferably comprise elements that most closely mirror the actual infectious particle or portion thereof. This should reflect the quasi-species genotypic and phenotypic variance noted in the intact host. The virions used for vaccine manufacture can come from any tissue source, but seminal, vaginal, and/or rectal tissue would be preferred.

### Isolation of Viral Nucleic Acids

Isolation of viral nucleic acid RNA or DNA, from infected tissue can be accomplished by a variety of well defined laboratory procedures. The initial steps, if viral DNA is to be isolated, consist of enzymatic or mechanical degradation of cell wall material, if present, and detergent lysis of cell membranes. After cellular disruption, proteins either viral or host derived are separated from nucleic acid.

Freshly harvested tissues and cells are ideal for isolation of nucleic acids. Storage of tissues and cells compromises nucleic acid integrity. If long term storage is needed either filter paper or freezing the DNA at −20° C. in TE buffer at a ph of 8 is recommended. The DNA storage medium should be free from water and contaminants. Long term storage of biological fluids such as urine and semen, although not preferable, can be accomplished at −20 to −80° C. (Bowien, et al., Ch. 2)

Two very simple techniques for isolating DNA from cells have been described: (1) incubation of cell lysates at high temperatures (for example 90° C. for 20 minutes); and (2) proteinase K digestion. Both techniques are limited in application and often are compromised by numerous contaminants. (Bowien, et al., Ch. 2)

Biological tissues may be made of uniform composition prior to nucleic acid separation using rotor-stator homogenizers. Alternatively, a mixture mill can disrupt and homogenize cells and tissues prior to nucleic acid separation. (Bowien, et al., Ch. 2)

The molecular structure, electrostatic character, and diffusion coefficient of RNA and DNA are quite similar. Therefore, many DNA isolation methods will be compromised by RNA impurities. Treatment with RNase A will remove RNA. RNase A solution should be heat treated prior to use to remove any contaminating substances with DNase activity. DNase-free RNase is also commercially available. RNase H can be incorporated into the DNA isolation procedures at various points, including the startup medium and/or final product. (Bowien, et al., Ch. 2)

Organic extraction methods consisting of phenol or phenol/chloroform mixtures are defined in the literature. (Bowien, et al. Ch. 2) The process of Southern Blotting is a further refinement used to detect HIV nucleic acids and consists of phenol/chloroform/isoamyl alcohol (25:24:1 ratio) extraction medium and is also described in the literature. Ribonuclease (RNase) can be added to digest the RNA in the preparation to isolate viral DNA. Further isolation of intact viral DNA from viral DNA fragments and host DNA can be accomplished by gradient centrifugation. (Michael, et al., Ch. 9 and 10)

“Salting-Out” methods of viral nucleic acids are another option. The cell lysate is exposed to a hypertonic medium which facilitates the precipitation of proteins and other contaminants. Centrifugation removes the precipitates and the viral DNA is recovered by a second step alcohol precipitation. DNA purity and quantity of yield is at times unpredictable with this method. (Bowien, et al., Ch. 2)

Centrifugation through a cesium chloride density/ethidium bromide gradient can separate viral DNA found in a cell lysate formed by alcohol precipitation. Centrifugation requires several hours and the DNA band is extracted with isopropanol to remove the ethidium bromide. The DNA is then precipitated with alcohol. This method yields high quality DNA but is not automated and therefore time consuming, relatively expensive and may not be applicable to large scale use due to human variability. (Bowien, et al., Ch. 2) Once isolated however, and found to be ideal in a vaccine formulation, nucleic acid modification can proceed to delete the targeted sequences.

Another method of isolation is through selective absorption of nucleic acids to silica in the presence of high concentrations of chaotropic salts. These include but are not limited to guanidine hydrochloride, guanidine isothiocyanate, sodium iodide and sodium perchlorate. This methodology effectively separates DNA from RNA but other cellular contaminants need to be washed away before DNA of high purity and quality can be eluted from the silica particles with a low-salt buffer. Silica based methodologies are offered by several companies as kits. (Bowien, et al., Ch. 2)

Anion-Exchange methods based on the electrostatic interaction between the negatively charged phosphates of the nucleic acid and the positively charged surface molecules on the substrate are used for viral DNA isolation. Utilizing solid-phase anion-exchange chromatography viral DNA will bind to the substrate under low salt conditions. Contaminants such as RNA and proteins are separated using medium-salt buffers. The DNA is then eluted with a high salt buffer and is of high quality relatively free of impurities. The eluted DNA is then recovered by alcohol precipitation and is suitable for genomic modification and amplification. (Bowien, et al., Ch. 2)

Filter paper impregnated with compounds of known DNA stabilization and isolation function can be used to store DNA before modification and amplification. Compounds that lyse cells, have bactericidal capacity, inhibit DNA degradation such as oxidation, and bind nucleic acids are on the filter paper. The DNA remains bound to the filter paper until eluted. This methodology allows for DNA storage at room temperature for several years without significant DNA damage or deterioration. (Bowien, et al., Ch. 2)

As mentioned, blood can be a source of genomic nucleic acid. Common anticoagulants such as heparin and EDTA can interfere with DNA isolation procedures and therefore should be avoided unless the blood is to be stored. QIAGEN® manufactures QIAamp® DNA blood kits for isolation of DNA from whole blood. Centrifugation and separation of whole blood fractions is not necessary with this procedure. In an alternative method, commercially available is the DNeasy® Tissue Kits and is based on silica-gel-membrane technology. QIAGEN® also manufacturers an anion-exchange technology for isolation of DNA in the Blood and Cell Culture DNA Kits. Finally, the QIAamp® UltraSens® Virus Kit from QIAGEN® can isolate HIV DNA from blood plasma and serum. (Botho Bowien, et al., Ch. 5)

Viral RNA may be preferable to DNA in certain embodiments. DNA is preferable if either is applicable due to the inherent instability of RNA. Prior to RNA isolation, host red blood cells and platelets should be removed from the viral source if blood is utilized. Red blood cells as mentioned above contain little nucleic acid and are poor sources for viral nucleic acid isolation. Removing erythrocytes simplifies RNA isolation since the ratio of rbcs/wbcs is 1000/1. The same methods to accomplish this procedure discussed with DNA isolation above apply but include but are not limited to: (1) hypotonic shock followed by centrifugation; and (2) Ficoll density-gradient centrifugation.

In general, cell poor material is therefore preferable to cell rich material if viral RNA isolation is the goal. This would limit laboratory procedure if the targeted viral RNA is extracellular. As discussed above with DNA extracellular nucleic acid may be non-infectious replication incompetent. The ideal source of viral RNA mirrors that of viral DNA, body fluids, transmitting the virus with sexual intercourse, the primary method of transmission of HIV today. The cellular derived RNA from such a cell poor body fluid would be more representative of replication competent infectious virions and would be preferable in the author's opinion. Viral RNA derived from a cell poor medium can be cell associated, cell free or combination of the two. The resulting viral RNA concentration, without regard to the source, can be anticipated to be low necessitating ultracentrifugation, ultra filtration or precipitation. (Bowien, et al., Ch. 6)

Cellular RNA from non-HIV infected tissues is comprised of three separate pools: (1) ribosomal RNA; (2) transfer RNA; and (3) mRNA. The mRNA carries the genetic information found in the DNA. The mRNA fraction is the smallest of the three, but is the necessary component for RNA based immunogenic composition or vaccine development. Of the total RNA in the typical mammalian cell, only 1-5% is mRNA. (Bowien, et al., Ch. 6) The RNA expression in a cell is quite variable. In HIV infected cells, a fourth pool of cellular derived RNA can be isolated consisting of a heterogenous mixture of viral RNA. Viral RNA in such cell lines is either single stranded, diploid (joined together only at specific sequences near the 5′ terminus), or found bound to its complementary DNA in a RNA/DNA duplex. Double stranded RNA molecules are also encountered assuming a helical structure more or less similar to the Watson Crick double helix. Single stranded RNA molecules include unspliced, singly spliced, or multiply spliced nucleic acid sequences. The unspliced RNA may or may not have a cellular derived 5′ cap and a 3′ polyadenylated (poly-A sequences) tail. In particular in a cell infected with the HIV virus mRNA content varies temporally, and is dependent on the expression of the Rev protein. After extraction of the viral RNA enrichment of the mRNA fraction can be accomplished by adding oligo(dT)-cellulose. This may be used to bind to and separate the poly(A) tails of eukaryotic mRNAs. This facilitates separation of the mRNA from the DNA, rRNA, and tRNA.

The process of sample harvesting and handling can influence mRNA production within seconds. Ideally the mRNA isolated for vaccine production should mirror the mRNA produced in vivo. Cell death, however, and enzymatic degradation of RNA by cellular and viral derived RNase enzymes can quickly destroy the mRNA fraction. Likewise, sample processing and handling can induce or down regulate the expression of certain viral genes. Therefore, mRNA should be stabilized prior to any nucleic acid isolation procedures. Rapid freezing in liquid nitrogen or with ethanol and dry ice have been used to stabilize mRNA with unreliable results.

Inactivation of cellular or viral derived RNases is preferred early in the laboratory process of RNA isolation. RNase enzymes are ubiquitous within the cell, generally do not require cofactors to function, relatively stable, highly efficient and often difficult to inactivate. Lysis of a cell to obtain viral nucleic acid subsequently releases the intracellular RNases. Chaotropic agents including guanidine isothiocyanate and guanidine hydrochloride immediately inactivate RNases. Also, digestion of contaminating DNA can be accomplished with DNase I. (Bowien, et al., Ch. 6) DNase I treatment can be performed at the beginning, middle, or end of any laboratory protocol involving RNA isolation but should usually follow treatment with RNases inactivating compound.

A mixture of mercaptoethanol, sarkosyl, and guanidine thiocyanate has been used to inactivate RNases and purify viral RNA from tissue specimens at a pH of 7.0. Sodium acetate at a pH of 4.0 and acidic phenol are then added allowing the RNA to be precipitated with alcohol.

RNA preservative compounds, such as RNAlater® RNA Stabilization Reagent are commercially available. This allows storage of the tissue sample before mRNA isolation for extended periods of time. Another example is the PAXgene® Blood RNA System for RNA stabilization and purification. This product prevents gene transcription. (Bowien, et al., Ch. 2)

If cell rich media are used for RNA isolation cell lysis with proteinase K in a vehicle containing an RNase inhibitor sodium dodecyl sulfate (SDS) is a relatively easy procedure. The DNA can be removed with DNase 1. Organic extraction followed by alcohol precipitation or well defined silica-based or anion-exchange methods will remove any excess contaminating DNase. Separation of viral RNA for genomic RNA can be accomplished by centrifugation or gel electrophoresis. (Bowien, et al., Ch. 6)

Alternatively, the above mentioned chaotropic agents not only inactivate RNases, but also disrupt cells. Organic extraction follows chaotropic extraction and involves one or more of the following defined technologies: (1) alcohol precipitation; (2) LiCl precipitation; (3) CsCl density gradients; (4) silica-based methods; (5) anion-exchange methods; and (6) hybrid selection. (Bowien, et al., Ch. 6)

HIV RNA conjugated to the HIV nucleocapsid protein is stable for approximately 2 to 3 hours. Quantification and amplification of HIV RNA is technically challenging, but can be accomplished with commercially available assays, such as the branched DNA assay from Chiron®, the Amplicor® RT-PCR assay from Roche®, and the NASBA amplification system by Organon-Teknika®. NASBA can selectively amplify RNA in compositions contaminated with DNA. NASBA can obviate at least one purification step separating the viral RNA from DNA. The fewer steps performed results in a streamlined laboratory procedure and a higher percentage of accurate genomic amplification. (Nelson Michael, et al., 1999, Ch. 16)

HIV RNA nucleic acids can be detected and isolated from a variety of tissues and in vitro cell lines with the process of Northern Blotting. Either a DNA or RNA probe can be employed with this technology, but more success has been noted with DNA. (Michael, et al., 1999, Ch. 10)

Commercially available reagents, such as Trizol®, are available for RNA extraction from tissue specimens. Silica based technology, such as the QIAamp® kits, can be used in cell lysates or cell free samples for RNA separation and purification. (Bowien, et al., Ch. 5)

Viral RNA can also be isolated and concentrated from stool specimens through a micro concentrator, such as the QIAamp® Viral RNA Mini Kit. (Bowien, et al., Ch. 5)

Other defined methods for isolation and stabilization of HIV RNA have been defined. These include but are not limited to cationic detergents such as Catrimox® used on whole blood samples, and the RNeasy® mini kit, which can be used to isolate viral RNA from blood after storage at room temperature for several months.

Viral RNA typically folds back on itself and assumes peculiar secondary structures. With HIV, viral RNA duplexes are performed by molecular bonding at a conserved region at the 5′ end. (Flint, et al., 2004, Ch. 7) Reverse transcription through these secondary and in the case of HIV tertiary and quaternary structures can be difficult. Commercially available reverse transcription enzymes, such as Omniscript® and Sensiscript®, are available for this purpose. (Bowien, et al., Ch. 6)

It would be reasonable to assume that the genotypic and phenotypic characteristics of a virus would be determined in part by the host cell type it invades and replicates in. Primary replication reservoirs of HIV include macrophages and T cells primarily found within the lymphoid tissue. In situ hybridization (ISH) allows the identification, concentration estimate and intracellular localization of specific nucleic acids, including DNA and mRNA as well as intracellular proteins. DNA, mRNA and protein can be detected simultaneously in an individual cell allowing the researcher to coordinate genomic content and genomic expression on an intracellular level.

ISH is relatively insensitive compared to the process of in situ PCR described below. ISH can detect mRNA concentrations as low as 20 copies per cell by those familiar with the art. Most laboratories performing ISH are more limited with a mRNA identification threshold defined as greater than 100 copies per cell. Although the process of in situ hybridization has been defined, a unified approach to all cell types with HIV is lacking. Nevertheless, the procedures are generally known.

In Situ Polymerase chain reaction allows the identification and amplification of intracellular DNA and RNA. This may prove to be preferable in vaccine production since many steps in nucleic acid isolation are obviated (streamlining laboratory procedures, facilitating nucleic acid purity and enhancing retrievable nucleic acid quantity) and the nucleic acid sequence identified will parallel that of infecting, replication competent virions. Most HIV virions produced are non-infectious and replication incompetent. In any system, in vitro or in vivo, contamination of infectious replication competent virions with non-infectious non-competent virions will inevitably result. An immunologic response directed to non-infectious replication incompetent virions may have no benefit or in the worst case scenario, adversely affect the host.

The concept of “original antigenic sin” has been well defined with influenza A, a segmented negative strand RNA virus in the family of orthomyxoviruses. The primary response of the host to a pathogenic organism blocks further immunologic response to that organism until an antigenically completely different strain infects the host. (Parham, 2005, Ch. 8) The concept of “original antigenic sin” may very well apply to other pathogens, including HIV. Optimally, an initial vaccine (subunit, live, conditionally replication competent, recombinant or otherwise) should closely parallel the actual infecting organisms and not defective virions, which may serve as an immunologic decoy thwarting an appropriate immune response by the host and block subsequent immune response to similar pathogens.

Utilizing one or more of the above mentioned sources for providing HIV nucleic acid isolation-modification and amplification would follow for composition or vaccine development. One embodiment of the present invention would further embrace the separation of infectious replication competent virions from non-infectious replication incompetent virions before isolation of the nucleic acids. This could be accomplished by isolating infected cells of the cell poor medium as the source(s) of HIV nucleic acid. Such isolation may be accomplished by centrifugation of bodily fluids. In situ hybridization and in situ PCR will then allow the identification and amplification of the preferred nucleic acids. In reverse transcription-in situ PCR can also be accomplished. mRNA fragments less than 1.5 kilobases can undergo RT-in situ PCR with technology available today. This would not allow the RT of the entire HIV genome which is 10 kilobases in length. However, overlapping or sequential mRNA fragments after in situ RT PCR can be ligated with DNA ligases to produce an intact HIV DNA genome suitable for modification and amplification. (Michael, et al., 1999, Ch. 18)

Reverse transcriptase enzymes are commercially available, such as Superscript II®, which lacks RNase H activity (degradation of single strand RNA in the reverse transcribed RNA/DNA heteroduplex) and is therefore more efficient at DNA amplification. It is capable of reverse transcribing relatively long mRNA molecules and can be used for routine RT amplification.

Annealing temperatures for reverse transcription and DNA amplification have been mathematically defined for in situ hybridization and in situ PCR. Re-annealing temperature parameters can also be defined with a thermocycler designed with a temperature gradient block for the rapid empirical determination of annealing temperatures block or the Touchdown PCR. (Michael, et al., 1999, Ch. 18)

The quantity of nucleic acids identified and amplified with in situ PCR and ISH is characteristically much less than that of solution based PCR methods discussed below. Investigators in the field have concluded that the rate limiting factor with ISH and in situ PCR is the difficulty primers have in traversing cell membranes. This may be overcome by several methods, including by way of example: (1) heat shock applied to cells, which temporarily increase membrane permeability to macromolecules; (2) coupling of primers to cell penetrating peptides (CPPs); and/or (3) a combination of (1) and (2).

### Methods for Quantifying DNA & RNA and Assessing Purity

Laboratory methods to assess RNA and DNA concentration and purity have been standardized and are quite similar. The concentration of RNA can be determined by measuring the absorbance at 260 nm (A₂₆₀) using quartz cuvettes, which allow UV light to pass with minimal distortion and absorption in a spectrophotometer. A pH of 7.0 throughout the procedure can assure validity and reproducibility. The ratio of absorption values at 260 and 280 nm provide an estimate of the purity of RNA. Kits are commercially available, such as the Oligotex® mRNA Kit, for quantifying mRNA. (Bowien, et al., 2003, Ch. 6; Nicholl, Ch. 3)

Quantifying DNA concentration and purity may also performed by a spectrophotometer performing a measurement of absorption at 260 nm in a quartz cuvette. Agarose gel analysis can also be employed for DNA quantification. As with RNA, the purity of DNA can be determined by measuring the A₂₆₀/A₂₈₀ ratio. (Bowien, et al., Ch. 7; Nicholl, 2002, Ch. 3)

Polymerase chain reaction can be used not only to amplify DNA and/or RNA sequences but also to remove primers, enzymes, salts, buffers, nucleotides and other contaminants. Kits such as the QIAquick® 96 PCR Purification Kit allows for PCR purification of DNA and is silica-gel-membrane based technology. An alternative method of purifying DNA is the MinElute® Reaction Cleanup Kit procedure. This procedure is also based on silica-gel-membrane technology. (Bowien, et al., Ch. 7)

Purification of RNA can be accomplished using RNase-free DNase I and is commercially available, such as the RNeasy® Kits and the QIAamp® RNA Blood Mini Kit for RNA purification, which are based upon silica-membrane, spin-column technology obviating the need for DNase treatment. (Bowien, et al., Ch. 6)

The actual sequence of a DNA molecule can be performed by two well defined methods in the literature. The Maxam-Gilbert sequencing method is based upon a set of nested fragments and involves radio labeling the DNA with ³²P at the 5′ end of each strand. The Sanger-Coulson (dideoxy or enzymatic) sequencing method utilizes a Klenow fragment of DNA polymerase and a primer to provide a 3′ terminus for the DNA polymerase. (Nicholl, Ch. 3)

Either of the above methods can be used for sequencing the intact viral nucleic acid, the modified nucleic acid used in the replication dependent virion as well as the nucleic acid sequence of the cleaved fragment. Furthermore nucleic acid sequence encoding the subunit component of the vaccine vector can be determined.

### Nucleic Acid Modification

Once the selected viral nucleic acid has been isolated, sequence modification can commence. As noted above, modification as used herein may include deletion and mutation as well. Either RNA or DNA can be utilized, but DNA would be preferable. DNA is more stable, easier to amplify ex vivo, and mutation of DNA may be accomplished more efficiently. An RNA template of the viral genome can also be used. The reverse transcriptase enzyme, an RNA-dependent DNA polymerase, produces a complementary strand of DNA from RNA. Alternatively the RNA can be modified by deletion of overlapping and/or non-overlapping segments. The PCR itself can introduce point mutations, deletions or insertions into DNA. (Flint, et al., 2004, Ch. 2)

Production of conditionally live virions may be undertaken by the use of bacterially or otherwise derived restriction enzymes to cleave the desired sequences out of the intact viral genome. To excise the genetic sequence of the targeted proteins, a complement of restriction enzymes can be used. In this process, the genetic sequence surrounding the codon of the targeted protein will be identified.

Restriction enzymes are produced by bacteria as a defense against infection by viruses. More than 200 restriction enzymes have been identified and are commercially available; about 100 of these enzymes are commonly used by researchers. Each restriction enzyme binds to DNA and recognizes a specific nucleotide sequence called a recognition sequence. The enzyme cuts both strands of the DNA within the recognition sequence in a specific cleavage pattern. This is followed by a purification step of the modified nucleic acid sequence.

The fragments generated by use of restriction enzymes may have blunt ends, 3′ protruding ends, or 5′ protruding ends. Modification to the cut DNA can be performed before the ends are re-annealed. For example, the enzyme terminal deoxynucleotidyl transferase (TdT) repeatedly and randomly adds nucleotides to any available 3′ terminus in a non-templated fashion. (Nicholl, 2002, Ch. 4) This includes protruding, blunt-ended and recessed 3′ termini. Once the two ends of the DNA sequence are linked back together (e.g., by addition of ligase), a knockout HIV virion may be created, which serves as the basis of the present invention. Other DNA modifying enzymes such as exonuclease, which degrade the 5′ and/or 3′ termini of DNA may also be employed for this purpose

Four additional useful nucleases (Bal 31, exonuclease III, deoxyribonuclease I [DNase I] and S₁-nuclease) are well defined in the literature, each differ in the location and mode of activity and provide the molecular biologist fine cutting tools of the trade. Phosphate groups can be added or removed from the termini of the DNA molecule. The enzyme alkaline phosphatase cleaves the terminal phosphate molecule of DNA and the enzyme polynucleotide kinase adds phosphate groups on to the DNA termini.

Another enzyme, terminal transferase (terminal deoxynucleotidyl transferase) repeatedly adds nucleotides to any open 3′ DNA terminus. This includes protruding, blunt-ended and recessed 3′ termini. After the targeted nucleic acid sequence(s) have been deleted from the viral genome the remaining DNA fragments can be joined into a functional molecule by the enzyme DNA ligase. This viral genome with deletion of sequences necessary for accessory protein (and/or structural and/or enzymatic) production can be amplified utilizing PCR or other method of sequence amplification.

To assure and enhance purity of the modified nucleic acid several steps can follow. These include but are not limited to centrifugation, gel electrophoresis, nucleic acid sequencing, and reverse transcription of the genomic sequence with identification of all proteins transcribed and translated. The latter process can be accomplished in a cell free nutrient broth or an in vitro cell culture such as polymorphonuclear blood cells or a continuous cell line such as HeLa cells.

Removal of the non-overlapping segment(s) of the targeted protein(s) will result in the transcription of truncated, non-functional protein(s). Only those targeted proteins will be adversely affected because each segment excised encodes just part of one protein. This will, nonetheless, disable and inactivate the HIV virions. The resulting virions will be replication incompetent and non-infectious by themselves. As discussed elsewhere, replication will require an exogenous source of the deficient protein(s). Removal of overlapping segments will adversely affect all the proteins partially encoded by the overlapping segments.

Using generally available techniques of molecular biology, DNA can be cut at precise target areas such as those sequences encoding for the non-overlapping portions of the vif, vpr, vpu, tat exon 1 and vpx codons. One or more mutations adversely effecting one or more structural, enzymatic or accessory proteins renders the virion incompetent. (Flint, et al., 2004, Ch. 20) These mutations can be one or more base substitutions, base deletions (contiguous or non-contiguous), or deletions of nucleotide sequence(s). In the context of the conditionally live virion, excision of non-overlapping and/or overlapping gene segments of the targeted protein(s) will be utilized. Point mutations in and of themselves do not allow sufficient safety parameters due to the propensity for back mutation to occur allowing the virus to become replication competent.

### Nucleic Acid Amplification

Solution-based PCR technology, primarily a method of nucleic acid amplification discussed below, does not differentiate the source of the viral nucleic acids. Furthermore, this technology is not dependent on the source of nucleic acids. In most applications, PCR is not a mechanism of nucleic acid identification, purification or isolation. The exception is in situ PCR (discussed above) which does allow intracellular nucleic acid identification, purification and isolation.

Polymerase chain reaction (PCR) enables the researcher to selectively amplify DNA sequences of any organism a million fold or more. The procedure relies on the choice of primers from two conserved regions of the viral genome. Most processes utilize the tRNA primer binding site located at the juxtaposition of the 5′ LTR and the gag nucleotide sequence and mRNA polyadenylation signal site at the R/U5 junction of the 3′ LTR. This amplifies the 9 kb of viral DNA encompassing all the coding regions for structural, enzymatic and accessory proteins and U3 and R domains of the 3′ RTR. Infectious pro-virions cannot be realized without the intact 5′ and 3′ LTR. Separate amplification of the LTR regions not included in the PCR reaction can be regenerated by separate amplification and DNA ligation can be utilized to produce an intact genomic sequence with both LTRs. (Michael, et al., 1999, Ch. 12; Nicholl, 2002, Ch. 7; Specter, et al.)

Other methods of nucleic acid amplification besides PCR have been defined and include, but are not limited to nucleic acid sequence-based amplification (NASBA) and transcription-mediated amplification (TMA). Additionally, strand displacement amplification (SDA), ligase chain reaction (LCR), cycling probe technology (CPT), and Cleavase invader assay are used for nucleic acid amplification. Detection of viral nucleic acids can be accomplished by these methods as well. Other laboratory procedures directed only at signal amplification without increasing the number of nucleic acid sequences have been defined and include but are not limited to enzyme immunoassay technologies (EIA), branched chain DNA (bDNA), and hybridization protection assay (HPA), and fluorescence resonance energy transfer (FRET) procedures. (Specter, et al.) Nucleic acid identification by signal amplification methodology can precede nucleic acid amplification. This can streamline laboratory procedures.

All amplification methods, regardless of procedure, are preferably performed with the cellular enzyme uracil-N-glycosylase (UNG). This reduces PCR carryover contamination. Pretreatment of the PCR reaction mixture with UNG for 10 minutes at room temperature cleaves and excises uracil residues from the DNA molecule. Heat inactivation then removes any residual UNG. This process ensures genomic purity. (Michael, et al., 1999, Ch. 15)

Classically the PCR process utilizes Taq polymerase which is derived from the thermophilic bacterium Thermus aquaticus which inhabits hot springs. (Nicholl, 2002, Ch. 7) Other similarly functioning polymerases are now coming available having enhanced speed and accuracy of genomic replication. (Michael, et al., 1999, Ch. 15) Other DNA polymerase enzymes include Pwo, Tth and HotTub DNA polymerase, which have been employed and often can be used when contaminants are present.

Real time PCR technologies such as probes and sequence detection systems can allow PCR isolation and amplification procedures to occur with minimal risk of laboratory contamination. (Bowien, et al., Ch. 8)

Nucleic acid amplification can be performed before or after nucleic acid modification and purification of modified nucleic acid sequence.

### Assembly of Conditionally Live Virions

The viral DNA or RNA after modification (e.g., excision of the designated sequences, other mutations, etc.) must be repackaged. This can be accomplished in a cell free expression system/medium or a cell culture. Within intact virions in vitro and in vivo, the process of reverse transcription starts to occur before viral fusion with the cellular membrane. Within the sub-viral particle, viral RNA is reverse transcribed by the reverse transcription enzyme which is included within the capsid core region. Therefore DNA, RNA or both can be the starting point for viral particle assembly. (Flint, et al., 2004, Ch. 4, and App. A)

The viral structure dictates an orderly, predictable sequence of self-assembly. The nucleocapsid protein is the foundation for the capsid protein. Likewise, the capsid protein is the scaffolding for the matrix protein. The matrix protein is the scaffold for the gp120/gp41 trimers. At the site of viral assembly, approximately 10% of the gag polyproteins (gag-pol) carry the translation products of the retroviral enzymes, protease (PR), reverse transcriptase (RT), and integrase (IN) at the 3′ or COOH terminus of the gag-pol protein.

Initially two strands of RNA are linked by specific sequences (dimerization initiation site and dimer linkage structure) in the 5′ LTR of the virus. This process occurs in vitro and initiates viral assembly. The linking of the two RNA strands facilitates conformational changes in the RNA that allow the next step to occur. The NC protein (p7) then coats the viral RNA. The accessory proteins, Vif and Nef are associated with the viral RNA necleocapsid complex and incorporated into the intact virion. Approximately 2,000 molecules of p7 are found in the intact virion on the surface of the viral genome. The association of the NC segments (p7) with the diploid viral RNA genome triggers its association with the p6 protein which has attached to it the Vpr (HIV-1 and HIV-2) and Vpx (HIV-2) accessory proteins. Approximately 2,000 p6 proteins are assimilated into each virion.

The viral encoded enzymes, RT, 1N, RNaseH and protease are then non covalently bound to the diploid viral RNA/p7/p6/Vpr/Vpx(HIV-2) complex. Approximately 10 copies of each of the enzymes is included in each virion. The viral capsid protein binds to a ubiquitous cellular protein cyclophilin A (CypA) which demonstrates cis-trans peptidyl-prolyl isomerase activity. Binding of CypA to p24 occurs at capsid sequence 87 His-Ala-Gly-Pro-Ile-Ala 92. A conformational change in the capsid protein occurs facilitating the next step.

The diploid viral RNA/p7/p6/Vpr/Vpx(HIV-2) complex is the foundation for assembly of the capsid (p17 or CA) protein with the CypA molecule. Approximately 2,000 molecules of p17/virion are needed to complete the next step. Only 200 CypA molecules are incorporated into each HIV virion. Ideally, the ratios of assembled viral and cellular derived proteins must mirror the final composition of the intact virion. The HIV-2 virion does not assimilate the CypA molecule and therefore this step is not necessary for assembly of HIV-2. The assembled capsid protein/CypA complex assumes a cylindrical shape and is the foundation for assembly of the matrix protein (p24).

The matrix protein forms an icosahedron around the capsid cylinder. Approximately 2,000 matrix protein monomers self-associate to form the icosahedron. This structure is the foundation for assimilation of the gp41 molecules. The crystal structure of MA is trimeric and trimerization of the MA structural protein appears to be a conserved of property of lentiviruses. (A. Cimarelli, et al.). The gp41 molecules self-assemble into homo trimers on the exterior surface of the matrix protein icosahedron. The gp120 molecule is the most exterior protein of the virion and is the last to be assembled into the virus. The gp41 trimer assumes a three dimensional structure and displays electrostatic properties that match the gp120 molecule. The gp41/gp120 interaction can be likened to a golf ball (gp120) sitting on top of a tee (gp41). Seventy two gp41/gp120 trimers form the exterior protein coat of the virus.

Viral RNA/protein interactions as well as viral protein/viral protein interactions and viral protein/host cellular protein are mediated by non covalent bonding such as van der Waals forces, hydrogen bonding and dipole-dipole moments. These intermolecular forces determine the order of virion assembly and final three dimensional structure of the virion.

At the cytoplasmic side of the plasma membrane of an HIV infected cell Gag polyprotein and Gag-Pol polyproteins accumulate in a 10 to 1 ratio. The protease enzyme cleaves these polyproteins during and after but not before the process of viral budding. Protease activity occurs after the pro-virion acquires a cellular derived envelope. It is documented that in each HIV virion approximately 2,000 copies of the p7 and p24 protein are assimilated. Approximately 90% of the p7 and p24 proteins are derived from the Gag polyprotein and approximately 10% from the Gag-Pol polyprotein. Cleavage of these polyproteins occurs during budding within an intact cellular derived enveloped and therefore the all the individual proteins derived must be assimilated into the intact virion.

By inference, 2,000 copies of p7 and p24, and approximately 1,800 are derived from the Gag polyprotein and 200 from the Gag-Pol polyprotein. At this point of virion assembly, loss of individual protein monomers is unlikely due to the cleavage of the polyproteins within the enveloped budding virus and the overall high efficiency of virion assembly. Therefore, it is reasonable to assume that 2,000 copies of p6 and p17 are included in each HIV virion. The Gag and Gag-Pol polyprotein encode in a 5′ to 3′ direction one copy of p17, p24, p7, p6 and with Gag-Pol one copy each of the viral enzymes. Thus, up to 200 copies of each of the viral enzymes may be incorporated into each virion. In a cell free expression system for self assembly of viral particles, the ratios of viral proteins including enzymes would reasonably be consistent.

The matrix protein facilitates both nuclear targeting of the preintegration complex and plasma membrane targeting of newly transcribed gag polyproteins. The matrix protein in the gag polyprotein binds with a cellular derived myristoyl moiety. This allows a directional change in the matrix protein. The Nef protein is also myristoylated during the process of viral assembly polarizing it to the cytoplasmic side of the plasma membrane. In the above methodology, incorporation of the myristoyl moiety into the matrix and Nef protein is to be avoided. The myristoyl moieties are added after viral entry into a targeted cell and are not components of the intact virion. The myristoyl moiety in an intact virion would not allow nuclear localization of the PIC. Therefore the cell free medium used for HIV virion production must be devoid of all myristoyl moieties or similar fatty acid substances. Enzymes that catalyze myristylation are also to be removed.

The matrix protein binds specifically to the internal cytoplasmic domain of gp41. The gp41 glycoprotein non-covalently attaches gp120. Fusion of the plasma membrane around the budding virion(s) initially releases an immature, non-infections virus particle. The viral protease enzyme then continues to cleave the gag and gag-pol polyproteins, resulting in an infectious particle.

Additionally, the self assembly process may be controlled by modulating the following:

pH

osmolality

temperature

relative ratio of viral proteins

order of viral proteins added

inclusion of facilitating or inhibitory non-viral substances

intensity, frequency and duration of light especially light in the ultraviolet range

Preferentially, the pH, osmolality, and temperature should reflect the intracellular environment: (pH=7), osmolality (=280 mosm), temperature=37° C. UV light particularly at the 260 nm band is to be avoided. At this wave length, conformational changes in both RNA and DNA are observed. Particularly, thymine dimmers occur in DNA as a result of exposure to UV light at 260 nm. The relative ratios of viral proteins should reflect the ratio of protein monomers in the intact virion. The order of viral proteins added depends on the desired end product, but in general to maintain the orderliness of the system, internal structural proteins are typically the starting point with the ending point being most external structural proteins. In general the sequence of the Env gene and the Gag gene in a 3′ to 5′ direction encode viral proteins mirroring this internal to external arrangement. Virally encoded enzymes as well as certain accessory proteins (Vpr in HIV-1 and HIV-2 and Vpx in HIV-2) are included within the capsid core but are not structural. Inclusion of these proteins is necessary for this composition or vaccine, since a conditionally live replication competent virion is contemplated. In a replication competent composition, viral encoded these enzymes and the above-mentioned accessory proteins are also necessary.

The production of the virion can be catalyzed by the virion encoded protease enzyme, which cleaves the Gag polyprotein and Gag-Pol polyprotein into the individual protein components in the temporally defined sequence that optimally facilitates intracellular viral production. Nef protein is also cleaved by protease during and after budding. Therefore, inclusion of this enzyme with the Gag polyprotein (or Gag polyprotein and Gag-Pol polyprotein in a 10-1 ration) is an alternative method of viral production (versus sequentially adding each protein).

Viral self-assembly is not an ATP or GTP consuming process. Consequently each step follows logically from the preceding step resulting in a state of lower entropy. Entropy is the number of possible arrangements of the elements in any system. It is a measure of randomness or dispersion. Without the consumption of energy, matter falls into structures with lower entropy. To maintain variability energy must be consumed and living cells divert much of their energy resources towards maintaining this dispersion/non-dispersion ratio. Viruses are not live structures. They do not produce or consume ATP or GTP but rely entirely on host cellular transcription and translational machinery.

Entropy, as it is generally considered, does not apply to viruses. Except for the most complex of all viruses. which may represent a bridge between viruses and bacteria, viral structures assume one of two possible low entropy states: icosahedral or helical. HIV exterior structure is an icosahedral structure characterized by twenty triangular faces, and twelve vertices, and can be viewed from a two fold, three fold or five fold rotational axis of symmetry. Although the gp120 and gp41 glycoproteins are the exterior or surface proteins of the HIV virus, the underlying matrix protein defines the icosahedral structure.

Icosahedrally symmetric structures are based upon a triangulation number, T, the number of structural units per face. The minimal number of subunits to self-assemble into an icosahedron is 60. With only 60 subunits each must be identical to produce an icosahedron. In this model T=1. If more than 60 units are found within the viral structural protein, each unit or subunit is found in a quasi-equivalent position, which is defined by the non-covalent bonding properties of the subunits. Although different structural environments may define a larger icosahedron, the non-covalent bonding properties of the subunits are similar (but not necessarily identical as seen in the simplest 60 subunit structure). Regularity and close fitting of molecules in any structure permits strong inter molecular structures, hydrogen bonding, dipole-attractions, and van der Waals forces.

The flexibility of the subunit protein(s) that comprise the exterior or interior structures confers another dimension to viral capsid self-assembly. Structural complementarities between contiguous capsid monomers as well as the coordinated electrical interactions define the final multi-subunit protein structure formed. Each subunit, therefore, can have multiple domains, each with its own three dimensional structure with each domain assuming a particular orientation to the other domains.

Therefore, the rule of triangulation numbers with some viruses may not seem to apply. With the consideration of separate flexible protein domains and each be considered as a separate structure, the triangulation number rule applies. Without energy expenditure viral assembly has to follow an orderly sequence to arrive at the structure of the lowest entropy. The crystal structure of MA is trimeric and trimerization of the MA structural protein appears to be a conserved of property of lentiviruses. (Cimarelli, et al.)

Structural, enzymatic, and accessory gene products necessary for virion production can be produced in vitro. Genetic transfer using a generic retroviral vector (RV) is a well documented method of gene transduction. Utilizing both the 5′ and 3′ LTRs, the packaging signal site (ψ site) and a polypurine tract a gene vector can be introduced into a cell culture such as yeast, E. coli or a continuous cell line such as HeLa. (Michael, 1998, Ch. 24) The genetic sequences encoding one or more marker proteins can be included in the retroviral vector as the exogene. The nucleocapsid (p7), p6, capsid, matrix, gp41 and gp120 structural proteins can be produced in a cell culture by gene transfer and spun off. Likewise, the genomic sequence for the retroviral enzymes and accessory proteins included within the intact virion can be introduced into cell culture and spun off.

The Tat and Rev proteins are not necessary for viral assembly, after budding are not structural proteins, and therefore the genetic sequences encoding these proteins do not need to be spliced into tissue culture to produce an intact virion. The Nef protein is packaged into HIV virions where the viral protease cleaves it. HIV proviral DNA synthesis is less efficient without the Nef protein. The Nef protein however is not necessary for viral replication, maturation, and budding. Preferably the protein components of the virus genetically encoded within the packaging lines will be added in a sequential fashion that parallels normal viral assembly and will include the Nef protein.

The orderly sequence of HIV virion assembly starting with the most internal structure and ending with the most exterior structure dictates the sequence of proteins and RNA to be followed in assembling conditionally live intact virions. Viral components in the appropriate ratios are added in one embodiment in a sequential fashion, mirroring the natural self-assembly process. Excess proteins are removed by centrifugation or other process before the next step. The virion is technically replication incompetent since the genomic information encoding one or more proteins necessary for replication in an intact host has/have been deleted.

In the above embodiment, a cell free system can be utilized. Therefore, an envelope will not be part of the viral structure. The envelope is acquired after virion assembly and before budding on the cytoplasmic side of the plasma membrane.

The hepatitis B vaccine is analogous in part to the above mentioned concept of a normally enveloped virus not dependent on the envelope for virion assembly, structure, and stability. The hepatitis B vaccine is produced in a yeast culture and contains one viral structural protein: the hepatitis B virus surface antigen. This structural protein spontaneously assembles into stable virus like particles. These particles are devoid of an envelope, yet are stable and immunogenic. Interestingly, hepatitis B encodes a reverse transcriptase enzyme similar to HIV. Hepatitis B is a DNA virus and HIV is an RNA virus.

Alternatively, the vaccine can be produced in a cell line, whether continuous or otherwise. The genome of the conditionally live virion, as mentioned above, can be spliced into a cell line (continuous or non continuous). This can be accomplished by restriction enzymes, as discussed above, in the production of the subunit component of this invention. The exogenous protein(s) not encoded in the nucleic acid sequence can be supplied to facilitate and control replication. Alternatively, the intact conditionally live virion with the modified replication protein exogenously added can be placed into an in vitro tissue culture. This duplicates the vaccine methodology described above in tissue culture. Replication of the virus will be controlled in part by the quantity and half life of the exogenously added protein. Alternatively, biologically active proteins or protein fragments of the modified gene sequence can be added to the in vitro tissue culture infected with the conditionally live virion.

Use of an in vitro cell line or culture to cultivate HIV leads to assembly of viral structures that will bear genotypic and phenotypic differences from HIV virions produced in the natural habitat or host (e.g., human being). This is a possible aspect of the second method for consideration in application. The second method, however, requires fewer steps and can proceed in a continuous cell culture ad infinitum if the appropriate nutrients are provided and the overall cell culture is conducive to continual cell replication.

### Production of Exogenous Protein (Subunit)

The conditionally live viral virion will require an exogenous supply of the deficient replication protein for replication. This replication protein can be produced in cell culture by gene transfer. Incorporation of the protein(s) into the host cell and the viral particle can be accomplished by coupling the protein to a cell penetrating peptide. A cell penetrating peptide (CPP) is an oligomer composed of 5-40 amino acids that is capable of passing through the plasma membrane of a cell and deliver intracellularly a variety of conjugated bioactive substances. A variety of mechanisms including endocytosis have been described in the literature to explain the mechanism of action of (CPPs). The delivered cargo can be covalently or non-covalently attached to the CPP. (Gellissen; Langel)

Ideally, the nucleic acid sequence encoding the one or more proteins deficient in the conditionally live virion are obtained from the intact nucleic acid of the same viral source. In one embodiment, the nucleotide sequence encoding for two or more contiguous proteins is cleaved out of the intact nucleic acid sequence. The overlapping and non-overlapping segments are removed, and therefore can be used in an in vitro expression vector to produce the complete amino acid sequence of these proteins. If only one protein is modified or deficient in the viral vector, then only the non-overlapping reading segment of that protein is removed. This non-overlapping nucleic acid sequence would not suffice for gene transfer. However, cleaving out the overlapping and non-overlapping sequence encoding one protein would permit gene transfer. The source of the genetic material in this instance may or may not be appropriate for the conditionally live viral vector. Supplying the proteins encoded by both the overlapping and non-overlapping segments into the tissue culture will result in viral replication and assembly. In an in vitro cell line in which one or more of the proteins encoded by the deficient nucleic acids in a particular virion are complemented by protein production by another virion in the same cell or in the same cell culture, assimilation of the deficient protein(s) into the virion will occur in a trans fashion facilitating viral replication and assembly.

With the isolated nucleic acid encoding one or more modified proteins, a suitable expression vector must be chosen. Classically plasmids, circular double stranded DNA molecules maintained in an extra chromosomal site within the cytoplasm of the cell, are used. Plasmids are small molecules containing an origin of replication to allow DNA to be copied, a selectable marker to visualize the vector, and one or more unique restriction endonuclease restriction sites enabling the insertion of the targeted DNA for large scale manufacturing. Plasmids generally are not necessary for cell survival, but often confer selective traits allowing the organism to survive under less than ideal conditions. Several naturally occurring plasmids have been defined and are available for gene transfer laboratory procedures. Other commercially available plasmids are the product of gene transfer procedures, and are not found outside of the laboratory.

Plasmids are found only in prokaryotic organisms in an environment that lacks nuclear membranes. Therefore transcription and translation occur simultaneously. Post transcriptional modification cannot occur in a prokaryote. Without post transcriptional and post translational modification, protein sequences encoded by viruses that infect mammals, such as HIV, may assume a structure in a prokaryote that differs significantly from that seen in the normal host. Therefore, these proteins produced by plasmids and prokaryotes, such as E. coli, may not be functional when assimilated into the normal eukaryotic host cell. Plasma derived viral proteins in a prokaryotic expression system may require additional modification steps before incorporation into an intact virion. (Desmond S. T. Nicholl, Ch. 5)

The eukaryotic organism most commonly used in genetic engineering is the yeast Saccharomyces cerevisiae. It is currently used for mass production of a vaccine for hepatitis B that is comprised of one structural protein of the virus, hepatitis B surface antigen. (Nicholl, Ch. 5) Saccharomyces cerevisiae post transcriptional and post translational modification of proteins closely parallels the post transcriptional modification of proteins in mammalian cells.

Bacteriophages have been used to transfer DNA into E. coli. Bacteriophages are viruses that infect bacteria. Other vectors for gene transfer consist of plasmid sequences joined to bacteriophage nucleic acid and are known as cosmids. This technology is well defined in the literature. (Nicholl, Ch. 5)

Eukaryotic cells allo post transcriptional and post translational modification of proteins. Therefore, they are preferred expression systems for viral proteins infecting mammals. In yeast, a variety of genetically engineered vectors including, but not limited to, yeast episomal plasmids, yeast integrative plasmids, yeast replicative plasmids, yeast centromere plasmids, and yeast artificial chromosomes have been described in the literature and can be used for producing HIV viral proteins in vitro. Furthermore bacteria artificial chromosomes (bacs) have also been defined. Bacs lack both post transcriptional and post translational modification machinery however. (Nicholl, Ch. 5)

Plasmids represent an ideal mechanism of extra chromosomal protein production but, for the most part, are limited only to prokaryotic organisms. The extra chromosomal location, as well as the ability for one cell to assimilate multiple identical plasmids, allows for continual protein production. The extra chromosomal location places the targeted nucleic acid sequence outside control of the organism chromosome. Transcription of a bacterial chromosome is under the control of promoters. Promoters, however, only control genetic sequences in cis. Promoters therefore do not control plasmid transcription.

Plasmid DNA introduced into mammalian cell cultures usually results in either degradative loss of the plasmid or integration of the plasmid into the host chromosome, and therefore is under control of the host chromosome. Most of the host chromosome is inactive in cellular transcription (heterochromatin). Insertion of a plasmid into or near heterochromatin will result in a loss of plasmid genetic expression. (Klug, et al.)

One exception to plasmid integration in mammalian cells has been defined. A plasmid containing the origin of replication of Epstein Barr virus, a virally encoded nuclear antigen of the Epstein Barr virus (EBNA-1), the binding site of EBNA-1, and a selectable marker provide the platform for such a plasmid. Removing the plasmid origin of replication and replacing it with random pieces of the human genome a plasmid vector can be produced that, upon entry into an in vitro mammalian cell culture, remains extra chromosomal in location and replicates autonomously. The nucleotide sequence for one or more HIV proteins can be spliced into this plasmid. The plasmid placed into a eukaryotic cell culture will be assimilated into the cytoplasm. Nuclear targeting of the plasmid will not occur. Transcription, translation and post translational modification of the viral genes will occur without nuclear control and therefore in a continuous fashion in the presence of EBNA-1 exogenously supplied. (RLP Adams) This is an appropriate mechanism for in vitro production of HIV viral proteins if a cell associated medium is anticipated

Isolation of a plasmid vector that is not commercially available may also be pursued. An appropriate culture medium for growing bacteria cell cultures for plasmid isolation is Luria-Bertani (LB) broth. Commercial kits, such as rapid extraction alkaline lysis (R.E.A.L.) Prep 96 Kits, permit the rapid isolation of plasmids, cosmids, bacs and phage artificial chromosomes. Silica based methods are also reliable methods of plasmid DNA isolation. (Bowien, et al., Ch. 3) The process starting from plasmid isolation to in vitro plasmid construction, purification and commercialization is outlined in the literature. (Botho Bowien, et al., Ch. 4)

Alternative methods of isolating individual protein components of the HIV virion can be used. The supernatant of an in vitro cell culture such as HeLa cells infected with HIV can be separated from the cell culture and individual viral proteins identified by gel electrophoresis, centrifugation, or other methods. Alternatively, the entire cell culture can be homogenized before separation of the individual HIV proteins. Both methods, although plausible, are not preferred due to contaminating material and the above mentioned genotypic and phenotypic differences between field isolates and in vitro HIV cell cultures.

Additionally chemical synthesis of proteins can be accomplished with a variety of amino acid sequencers.

Ways of constructing vectors are known to those skilled in the art (e.g., as illustrated by U.S. Pat. No. 7,132,271). Examples include using chemically or enzymatically synthesized DNA, fragments of the viral cDNA or targeted genes. Additionally, transfection of a cell culture is carried out by standard methods, for example, the DEAE-dextran method (McCutchen and Pagano), the calcium phosphate procedure (Graham et al), or by any other method known in the art, including but not limited to microinjection, lipofection, and electroporation. (Sambrook et al.) Transfectants having deficient replication or other activity are selected. For ease of selection, a marker gene such as neomycin phosphotransferase II, ampicillin resistance or G418 resistance, may be included in the vector carrying the antisense or mutant gene. When a marker gene is included, the transfectant may be selected for expression of the marker gene (e.g. antibiotic resistance), cultured and then assayed for the targeted activity.

In a host coinfected with two or more strains of HIV, circulating recombinant forms consisting of nucleotide segments of different viruses have been noted to evolve. Also noted is a codependency of non-viable virions to encode proteins that complement the deficient proteins each encodes, resulting in the replication and propagation of one or more otherwise non-viable virions. (Flint, et al., 2004, Ch. 20) A parallel exists between eukaryotic and HIV virions which are diploid. Eukaryotes possess a nuclear membrane, and typically have a diploid number of chromosomes. Therefore, a deficient protein encoded on one chromosome may not affect the viability of the organism if its complementary chromosome encodes a non-deficient protein.

HIV is a diploid virion (unlike most viruses which are haploid), and like eukaryotic organisms, the nucleotide sequences of the RNA strands do not have to be and frequently are not identical. In a cell infected with more than one strain of HIV, multiple opportunities exist for one strain to circumvent the defective proteins encoded by one or more other strains. The greater the number of different strains coinfecting a cell the greater the opportunity is for the propagation of non-viable strains. This explains, in part, that viral mutation assures viral survival.

The present invention builds on the above observed phenomena, which is a characteristic of HIV viral evolution. A virus genome defective in one or more proteins will become viable if those defective proteins are provided by another source. In one embodiment of the present invention, multiple viral strains will have the same targeted nucleic acid sequence removed, as described above.

An alternative in vitro methodology of conditionally live virion production involves the co-infection of a tissue culture with a first conditionally live virion and a second virion (conditionally live or otherwise) that includes the nucleic acid sequence spliced out of the first conditionally live virion. This would enable viral replication. However, a recombinatorial event in such a culture is likely to occur, potentially allowing a replication competent vector to emerge.

Some accessory viral proteins, including Vpr, Vif and Vpx (and Vpx in HIV-2), are found within the intact virion. These may sustain one or more rounds of viral replication and may be of sufficient quantity to generate an appropriate Th-1 response with immunologic memory and consequent immunity in a conditionally live virion deficient in the nucleic acid sequence(s) for Vpr, Vif, Vpx, or combination. Once the supply of deficient proteins is exhausted, replication ceases. Therefore, conditionally live virions deficient in the nucleic acid sequence(s) for Vpr, Vif, Vpx, or combination may or may not require exogenous protein supplementation in some circumstances. The number of replication cycles of the virus is ultimately still regulated and controlled by the quantity and half life of the deficient protein(s), but in this method no exogenously added proteins may be involved.

In one embodiment of the present invention, a knockout virion for the vpr sequence is targeted and removed as described above. But because each HIV viral particle assimilates approximately 100 copies of the Vpr protein, exogenously added Vpr protein may or may not be necessary. (Cohen, et al., Ch. 16) Once the supply of Vpr proteins is exhausted, viral infectivity, virulence, and replication will be seriously compromised. The intact immune milieu of a healthy host will mount an appropriate response and eradicate the intracellularly replicating virus.

It is possible for the vaccine or composition to be administered as the pure or substantially pure virion plus exogenously added protein, or as a pharmaceutical formulation or preparation, optionally with adjuvants or other compositions.

The formulations to be used in the practice of the present invention, both for veterinary or human use, comprise knock-out virions plus exogenously added replication protein, as described above, together with one or more pharmaceutically acceptable carriers and optionally, other therapeutic ingredients. Protein carriers must be “pharmaceutically acceptable” in the sense of being compatible with the other ingredients of the formulation and not deleterious to the recipient thereof. The coupling of protein carriers (e.g., complement proteins) is known within pharmacology.

Desirably, the formulation should not include other substances with which the HIV virion is known to be incompatible. In accordance with current pharmacological standards, the methods include the step of bringing into association the conditionally live virion and exogenously added replication protein with a carrier which may constitute one or more accessory ingredients. Formulations suitable for administration by injection conveniently comprise sterile aqueous solutions of the vaccine, which solutions are preferably isotonic with the blood of the recipient. Such formulations may be prepared to produce a pharmacologically acceptable sterile aqueous solution.

In one embodiment of the present invention, the deficient protein will be coupled to a cell penetrating peptide such as Penetratin, a fragment of the Tat protein (amino acid 48-60) Transportan, Signal sequence-based peptides, Arginine polypeptides, pVEC and or Amphiphilic model peptides. Coupling such as this is well known by those in the field and will facilitate plasma membrane passage into the host cells of the targeted deficient protein. (Ulo Langel)

In another embodiment, the present invention builds on the knowledge that ubiquitinated proteins have been correlated with a variety of cellular functions including but not limited to the processing and presentation of antigens to T cells. (Krauss) The exogenously added proteins can be poly ubiquitinated. To facilitate entry into the proteasomal pathway, the intact virion may also be poly ubiquitinated. Therefore conjugating the exogenously supplied viral proteins and/or conditionally live virion with ubiquitin will direct exogenously added protein and/or conditionally live virion to the proteasomal pathway, resulting in an MHC-I based Th-1 immune response to one or more epitopes on that protein and/or viral vector. Within the scope of the present invention are embodiments in which a portion of the exogenous protein is ubiquitinated; for example, half of the exogenous protein may be ubiquitinated.

### ALTERNATIVE EMBODIMENTS BASED ON CLEAVED NUCLEIC SEQUENCES

Any possible combination of sequence excision and ligation is anticipated so long as a conditionally live virion is created. Any accessory or regulatory protein compromised by sequence excision can be provided exogenously allowing viral replication to proceed intracellularly in a “normal” but limited fashion. Presently fifteen (15) conditionally live virions with HIV-1 and 15 conditionally live virions with HIV-2 are described, which are purely exemplary and are not meant to be utilized to limit the scope of this invention.

By way of example and in the simplest embodiment, the overlapping and non-overlapping genomic sequence encoding vif, vpr, tat (exon 1), vpu (HIV-1), and vpx (HIV-2) can be excised individually, or in combination with another, using restriction enzymes. The overlapping segments of vif with pol and vpu with env would not be removed, leaving these genes and the proteins they encode intact. Utilizing this knockout system, five separate proteins (Vif, Vpr, Tat (exon 1), Rev exon 1 and Vpu in HIV-1 or Vpx in HIV-2), which may or may not be included in the intact virion capsule can be exogenously applied.

In an alternative embodiment, an immunogenic composition devoid of the non-overlapping tat exon 1 genomic sequence with vpr is used. The entire Tat protein encoded by exon 1 and 2 is then exogenously supplied as part of the immunogenic composition or vaccine. Any formulation in which the tat exon 1 nucleotide sequence is removed will also result in removal of rev exon 1 nucleotide sequence. Rev exon 1 completely overlaps with tat exon 1 in most viral isolates. Therefore two proteins would need to be added to the viral composition, Tat and Rev. Preferably the entire Tat and Rev protein will be added which is encoded by two separate exons. Tat exon 2 completely overlaps rev exon 2 and both completely overlap env. In yet another embodiment an immunogenic composition devoid of the rev exon 1 genomic sequence is excised and the entire Rev protein encoded by exon 1 and 2 as well as the entire Tat protein could be exogenously supplied.

The intron sequence located between the 3′ terminus of the tat or rev exon 1 and the 5′ terminus of the vpu protein preferably would not be spliced out of any of the above or below mentioned vaccines. The polypurine tract important in reverse transcription of the HIV RNA genome is found within this intron sequence. In HIV-2 a similar sequence is found between the 3′ terminus of the vpx nucleotide sequence and the 5′ terminus of the vpr nucleotide sequence. In a likewise fashion splicing out this sequence would not be preferable.

By excising only one non-overlapping gene segment, four separate conditionally live virions for HIV-1 and four for HIV-2 can be developed. The proteins encoded by the truncated compromised genes can be exogenously administered.

If two non-overlapping gene segments are excised, six possible conditionally live virions are possible. The replication proteins encoded by the excised genomes can be exogenously supplied. If the genomes for two sequential proteins are excised, then the overlapping segments of the two may be excised as well. This would increase the safety and simplicity of design and manufacture, as only two “cuts” of the viral nucleic acid sequence would be needed, instead of four—and only one re-annealing process instead of two. That is, excision of non-sequential non-overlapping gene segments will generally require more “cuts” and more re-annealing.

Excising three non-overlapping gene segments yields four possible conditionally live virions. The proteins encoded by the excised genome can be exogenously supplied.

By excising all four non-overlapping genomic segments, one live virion results. Therefore, fifteen separate conditionally live virions may be created with HIV-1 and fifteen for HIV-2. The following list delineates the partially and/or completely excised genomic sequences of potential compositions.

HIV-1:

vif

vpr

tat exon 1

vpu

vif and vpr

vif and tat exon 1

vif and vpu

vpr and tat exon 1

vpr and vpu

tat exon 1 and vpu

vif, vpr, and tat exon 1

vif, vpr, and vpu

vif, tat exon 1, and vpu

vpr, tat exon 1, and vpu

vif, vpr, tat exon 1, and vpu

HIV-2

vif

vpr

tat exon 1

vpx

vif and vpr

vif and tat exon 1

vif and vpx

vpr and tat exon 1

vpr and vpx

tat exon 1 and vpx

vif, vpr and tat exon 1

vif, vpr, and vpx

vif, tat exon 1 and vpx

vpr, tat exon 1 and vpx

vif, vpr, tat exon 1 and vpx

### Administration and Adjuvants

The immunogenic composition or vaccine may be administered via erythrocyte-mediated micro injection. Erythrocytes are lysed in a hypotonic solution in vitro. The vaccine is added to the solution. The red blood cell membrane is very porous in the hypotonic solution and allows large proteins from the extracellular milieu to enter the cell. (Doherty, et al.) The red cells are then placed into a solution of normal tonicity (0.9% NaCl). The damaged red cells are sequestered and degraded within the spleen and liver. Antigen presenting cells in both organs, particularly the Kupffer cells lining the liver sinusoids will uptake the foreign material and present it to the appropriate T cells.

In another embodiment for vaccine administration, the intact skin, an organ of the body with minimal immunologic activity can become an effector organ of the immune system if its barriers are breached. Two to three days prior to vaccination, the area to receive the vaccine will be mechanically shaved creating a superficial abrasion. This will trigger effector cells and proteins of both the innate and acquired immune response to sequester at the injured site priming it for vaccine. Exposure to UV light which induces immunosuppression is to be avoided. The vaccine will be administered by the intradermal route into the abraded skin. This will result in an anatomically defined hierarchal immune response closely paralleling lymph node architecture. This is preferred method of vaccine administration by the inventor.

The preferred area of vaccination would be the upper medial thigh. The lymphatic drainage from this area is directly to the inguinal lymph nodes. The number of inguinal lymph nodes varies from 12 to 20 in number and not only filter the lymph from the lower extremity but also the lymph from the external genitalia, perineum, buttock and lower anal canal. (Ben Pansky) These are the sites of initial HIV infection and propagation in sexual transmission of the disease.

The present invention further contemplates that adjuvants or other compositions intended to boost the immune response to a vaccine may be added to all the above vaccine cocktail. Such adjuvants preferably are in a form to bind to the cocktail. Such compositions may include, but are not limited to, polysaccharides composed of at least one molecule of mannose, teichoic acid, zymosan, the polysaccharide capsule of cryptococcus neoformans serotype C, Protamine, heparinase, cobra venom factor in a form adapted to enhance production of C3b, cobra venom factor in the form of dCVF, Nickel in a form adapted to enhance C3 convertase activity, or sulfated polyanions. The operation of these adjuvants has been previously described in U.S. Pub. No. 20050112139, which is hereby incorporated by reference. Exemplary additional adjuvants may include:

Additional Adjuvants
(a) Heat shock proteins (HSP): HSP90 associates with several different intracellular protein chaperones to form multimeric proteins. Inhibitors of HSP90 results in rapid ubiquitination and Proteasomal degradation of their associated proteins, including intracellular pathogens or their subunits. (Hoffman, Ronald) HSP60 & HSP70: Activate immune cells, such as macrophages and dendritic cells. (Kaufmann, 2004, Ch. 13)
(b) Type III repeat extra domain A of fibronectin: Activate immune cells through recognition via TLR4.
(c) Low-molecular weight oligosaccharides of hyaluronic acid: Activators of dendritic cells also mediated by TLR4. (Kaufman, 2004, Ch. 13)
(d) Polysaccharide fragments of heparin sulfate: Induce maturation of dendritic cells via TLR4. (Kaufmann, 2004, Ch. 13)
(e) Fibrinogen: Induces chemokine production in macrophages through TLR4. (Kaufmann, 2004, Ch. 13)
(f) Lipopolysaccharides (LPS): The most powerful immunostimulator among microbial components. (Kaufman, 2004, Ch. 13)
(g) Phosphorylcholine (PC): a major antigenic structure found on gram positive bacteria. PC is bound by natural IgM antibodies as well as CRP (C-reactive protein). PC can therefore activate the complement system and enhance the innate immune response. The binding of CRP to PC is calcium dependent. The binding of PC to natural IgM antibodies is not calcium dependent.
(h) Uric Acid (UA): Human cells undergoing apoptosis, necrosis, or other form of cell death release a variety of non-specific danger signals, one of which is uric acid. Uric acid stimulates dendritic cell maturation and enhances the responsiveness of CD8 T cells to antigens. UA is a naturally occurring endogenous adjuvant. The administration of UA with a vaccine will enhance the efficacy of the vaccine.
(i) IgGI and IgGIII antibodies specific for either component of the vaccine (either component): Natural killer (NK) cells are characterized by an Fc receptor known as CD16 or FcγRIII which is specific for IgGI and IgGIII. By conjugating the vaccine with these antibodies the antibody-dependent-cell-mediated cytotoxicity (ADCC) of the NK cells will be enhanced. (Parham, 2003, Ch. 7) These receptors are also found on dendritic cells (DC). NK and DC cells undergo a process of mutual priming and in the case of dendritic cells maturation occurs as a result of this “cross talk”. DC cells are a bridge between the innate and acquired or adaptive immune system. Therefore IgGI and IgGIII antibodies affixed to a vaccine will opsonize the vaccine by enhancing the response of NK cells and DC cells, as well as facilitating their cooperation in dealing with a potential pathogen or vaccine. (Ferlazzo, et al.; Cooper, et al.; Chiesa, et al.)
(j) Complement Proteins: A variety of complement proteins, particularly C3b opsonize immunogens to which they are attached. This will enhance both the innate and adaptive immune response. (Hoffman, et al)

### CONCLUSION

The analysis and development of the immunogenic composition should incorporate a wide range of doses of inactivated particulate for evaluation. Animal trials should consider differences in size, species, and immunological characteristics; it is anticipated that immunological differences between humans and animals may relegate animal trials to toxicity analysis. Clinical trials will involve at least the standard three phase model, ranging from safety and dosage in a small population, safety and immunogenicity in a second phase of several hundred volunteers, to a large scale effectiveness phase. The clinical trials should include appropriate exclusionary criteria as is customary, such as exclusion for other immune suppression conditions, pregnancy, active drug use, etc.

In addition to administration routes described in detail above, administration may be made in a variety of routes, for example orally, transbucally, transmucosally, sublingually, nasally, rectally, vaginally, intraocularly, intramuscularly, intralymphatically, intravenously, subcutaneously, transdermally, intradermally, intra tumor, topically, transpulmonarily, by inhalation, by injection, or by implantation, etc. Various forms of the composition may include, without limitation, capsule, gel cap, tablet, enteric capsule, encapsulated particle, powder, suppository, injection, ointment, cream, implant, patch, liquid, inhalant, or spray, systemic, topical, or other oral media, solutions, suspensions, infusion, etc. Because some of the first targets for infection with HIV are epithelial cells and Langerhans cells in the skin and rectal and vaginal mucosa, then a preferable embodiment of delivery is dermal combined with rectal and/or vaginal suppositories. HIV is contracted predominantly by rectal and vaginal intercourse. Therefore rectal and/or vaginal suppository administration of the vaccine would be a preferred administration methodology. In addition, the present invention may be combined with other therapeutic agents, such as cytokines, including natural, recombinant and mutated forms, fragments, fusion proteins, and other analogues and derivatives of the cytokines, mixtures, other biologically active agents and formulation additives, etc. Those skilled in the art will recognize that for injection, formulation in aqueous solutions, such as Ringer's solution or a saline buffer may be appropriate. Liposomes, emulsions, and solvents are other examples of delivery vehicles. Oral administration would require carriers suitable for capsules, tablets, liquids, pills, etc, such as sucrose, cellulose, etc.

Thus, in conclusion, the present invention is based on a conditionally live virion; that is, a virion modified to be otherwise replication incompetent is enabled to be replication competent for a limited time upon the addition of exogenous protein, which substitutes for protein that is unavailable due to the modification (or deletion) of the corresponding genetic sequence encoding that protein in the viral genome. A virus by definition is not a live or dead structure. It is best characterized as being replication competent or replication incompetent. In this invention, a live virus refers to a replication competent vector. One aspect of the present invention is an immunogenic composition comprising a viral DNA or RNA representing a complete viral genome in which at least one replication protein gene or corresponding mRNA has been modified to render the viral DNA or RNA replication incompetent; this modified viral DNA or RNA is then encapsulated by viral proteins that self assemble in a cell free expression system, forming a conditionally live virion. The method for producing this conditionally live virion includes the steps of providing at least one viral DNA or RNA molecule representing a complete genome, amplifying the viral DNA or RNA, modifying the viral DNA or RNA in at least one replication protein gene or corresponding mRNA, collecting the amplified and modified viral DNA or RNA, repackaging the collected DNA or RNA in a cell free expression system suitable for self assembly of viral particles, and collecting a desired quantity of the resulting conditionally live virions. An alternative method for producing this conditionally live is using a traditional cell culture system. In this method, a virion modified in at least one replication protein gene or corresponding mRNA may be cultured under conditions suitable for viral replication with the addition of exogenous protein corresponding to the at least one replication protein gene or corresponding mRNA. Therefore, a fourth aspect of the present invention is formulating a vaccine using the replication incompetent virion in combination with whole viral proteins, protein fragments, protein derivatives, or combinations thereof. A vaccine created by either method will have three fold immunogenic properties that are elicited by 1) the whole intact replication incompetent virus; 2) the conditionally live virion temporally resuscitated by addition of protein supplements; and 3) the protein supplement itself acting as a subunit vaccine. An added feature of a vaccine formulated with the conditionally live virion created in the cell free system is that no vector is present to contribute to the elicited immunogenic response of the vaccine when administered.

The above examples should be considered to be exemplary embodiments, and are in no way limiting of the present invention. Thus, while the description above refers to particular embodiments, it will be understood that many modifications may be made without departing from the spirit thereof.

### BIBLIOGRAPHY

Aguzzi A, S. Brandner, U. Sure, et al., Brain Pathology, 1994, 4:3 20
Bour, Stephan, et al., 2003, The HIV-1 Vpu Protein: A Multifunctional Enhancer Of Viral Particle Release, Microbes And Infections, Vol. 5, pp. 1029-1039
Bowien, Botho, et al., 2003, Nucleic Acids Isolation Methods, Ch. 2, pp. 7-19; Ch. 5, pp. 53-59; Ch. 6, pp. 61-80; Ch. 7, pp. 81-94
Busby, S, et al., J. Mol Biol 154:197 209 (1982)
Campbell, Mary K., et al., 2006, Biochemistry, 5^{th} Ed., Ch. 12, pp. 320-321
Camper, S. A., et al., Biology of Reproduction, 1995 52:246 257
Chiesa, Mariella Della, et al., Pathogen-Induced Private Conversations Between Natural Killer and Dendritic Cells, Trends in Microbiology, Vol. 13, No. 3, March, 2005
Cimarelli, A. et al., 2002, “Biomedicine And Diseases: Review Assembling The Human Immunodeficiency Virus Type 1” Cellular And Molecular Life Sciences, Vol. 59, pp. 1166-1184
Cohen, P. T., et al., The AIDS Knowledge Base, 3^{rd} Ed., Ch. 16, pp. 153
Cooper, Megan A. et al., “NK Cell and DC Interactions,” Trends in Immunology, Vol. 25, Iss. 1, 2004, pp. 47-52
Deeks, Steven, The Medical Management of Aids, Ch. 6 (6th ed. 1999)
Deng, W. P., J. A. Nickoloff, Analytical Biochemistry, 200:81 88 (1992)
Doherty, F. J., et al., 1992, Intracellular Protein Degradation, Ch. 2, pp. 9-14
Ferlazzo, Guido, et al., 2004, NK Cell Compartments and Their Activation by Dendritic Cells, J of
Flint, S. J., et al., 2000, Principles of Virology, 2^{nd} Ed., App. A, pp. 836
Flint, S. J., et al., 2004, Principals of Virology, 2^{nd} Ed., Ch. 1, pp. 6; Ch. 2, pp. 51-53; Ch. 4, pp. 83-125; Ch. 7, pp. 217-250; Ch. 20, pp. 763-768; App. A, pp. 835-837
Gellissen, Gerd, 2005, Production of Recombinant Proteins, Ch. 2, pp. 7-37 and Ch. 11, pp. 233-251
Graham, et al., 1973, J. Virol. 33:739 748
Hoffman, Ronald, et al., 2005, Hematology: Basic Principals and Practice, 4^{th} Ed., Ch. 43, pp. 720-733; Ch. 55, pp. 80-982
Hout, David R., et al., 2004, Vpu: A Multifunctional Protein that Enhances the Pathogenesis of Human Immunodeficiency Virus Type 1, Current HIV Research, VI. 2, pp. 255-277
Immunology, Dec. 1, 2003, pp. 1333-1339
Immunodeficiency Virus Type-1 Tat Protein, Virology, Vol. 257, pp. 502-510
Kaufmann, Stefan, H. E., 1997, Host Response to Intracellular Pathogens, Ch. 3, pp. 37-45
Kaufmann, Stefan, H. E., 2004, The Innate Immune Response to Infection, Ch. 13, pp. 260
Klug, William S. et al., 2006, Concepts of Genetics, 8^{th} Ed., Ch. 4, pp. 66-99
Krauss, Gerhard, 2003, Biochemistry of Signal Transduction and Regulation, 3^{rd} Ed., Ch. 2, pp. 101-113
Langel, Ulo, 2002, Cell-Penetrating Peptides Processes and Applications, Ch. 1-8, pp. 1-162
Langel, Ulo, 2007, Handbook of Cell-Penetrating Peptides, 2^{nd} Ed., Ch. 1, pp. 5-23
Lee, et al., Virology 192:380 385 (1993)
Levinson, Warren, Medical Microbiology & Immunology, 2004, Ch. 36, pp. 237-243
McCutchen and Pagano, 1968, J. Natl. Cancer Inst., 41:351 357
Michael, Nelson, et al., 1998, HIV Protocols, Ch. 24, pp. 227-230
Michael, Nelson, et al., 1999, HIV Protocols, Ch. 1, pp. 3-10; Ch. 8, pp. 51-57; Ch. 9 & 10, pp. 61-81; Ch. 12, pp. 89-98; Ch. 17, pp. 151-164; Ch. 18, pp. 165-196; Ch. 19, pp. 185-196; Ch. 24, pp. 227-230; Ch. 35, pp. 323-327
Nicholl, Desmond S. T., 2002, An Introduction to Genetic Engineering, 2^{nd} Ed., Ch. 2, pp. 11-26; Ch. 3, pp. 27-41; Ch. 4, pp. 43-53; Ch. 7, pp. 115-130
Pansky, Ben, 1996, Review of Gross Anatomy, 6^{th} Ed., Ch. 6, pp. 497-254
Parham, Peter, 2003, The Immune System, 2^{nd} Ed., Ch. 7, pp. 204-205
Parham, Peter, 2005, The Immune System, 2^{nd} Ed., Ch. 3, pp. 67-96; Ch. 8, pp. 273-274
Parrish, John A., et al., 1983, Photo Immunology, Ch. 6, pp. 95-130
Primate Lentivirus Complete Genomes, http://hiv-web.lanl.gov/content/hiv-db/HTML/2005compendium.html
RLP Adams, 1991, DNA Replication, Ch. 4, pp. 49-62
Rubartelli, Anna, et al., “HIV-1 Tat: A Polypeptide for all Seasons”, J. Immun. Today, Vol. 19, Issue 12, p. 545 (1998)
Sambrook, et al., Molecular Cloning: A Laboratory Manual, 2nd Edition, (1989)
Specter, Steven et al., 2000, Clinical Virology Manual, 3^{rd} Ed., Ch. 17, pp. 169-179 and Ch. 19, pp. 188-197
Wagner, Edward K., et al., Basic Virology, 1999, Ch. 8, pp. 102-108
Wang, Zhongde, et al., 1999, Activation of Bcl-2 Promoter-Directed Gene Expression by the Human, 1999, Vol. 257, pp. 502-510

What is claimed is:

1. A method for the production of at least one conditionally live virion, comprising the steps of:
a. providing at least one viral DNA or RNA molecule representing a complete viral genome;
b. amplifying the viral DNA or RNA;
c. modifying the viral DNA or RNA in at least one replication protein gene or corresponding mRNA;
d. collecting amplified and modified viral DNA or RNA;
e. repackaging the collected viral DNA or RNA into a cell free expression system suitable for self-assembly of viral particles as conditionally live virions; and
f. collecting at least one conditionally live virion.

2. The method of claim 1, wherein said conditionally live virion is deficient in replication ability relative to a corresponding virion lacking a modification to its viral DNA or RNA.

3. The method of claim 1, wherein said viral genome is an HIV virus genome.

4. The method of claim 1, wherein said method further comprises preparing a viral vaccine from said collected at least one conditionally live virion.

5. The method of claim 1, wherein said method further comprises the steps of formulating a viral vaccine from said collected at least one conditionally live virion and adding to said viral vaccine a predetermined quantity of exogenous replication protein(s) corresponding to said at least one modified replication protein gene or corresponding mRNA, thereby enabling limited replication of said at least one conditionally live virion.

6. The method of claim 1, wherein said method further comprises the steps of preparing a viral vaccine from said collected at least one conditionally live virion and adding to said viral vaccine a predetermined quantity of exogenous replication protein(s) corresponding to said modified replication protein gene or corresponding mRNA, wherein said exogenous replication protein(s) is a biologically active fragment or derivative of said at least one modified replication protein gene or corresponding mRNA thereby enabling limited replication of said at least one conditionally live virion.

7. The method of claim 1, wherein said method further comprises the steps of preparing a viral vaccine from said collected at least one conditionally live virion and adding a pharmaceutically acceptable carrier.

8. The method of claim 1, wherein said method further comprises the steps of preparing a viral vaccine from said collected at least one conditionally live virion and adding at least one pharmaceutically acceptable adjuvant.

9. The method of claim 1, wherein said method further comprises the steps of preparing a viral vaccine from said collected at least one conditionally live virion and adding at least one pharmaceutically acceptable adjuvant selected from the group consisting of, polysaccharides composed of at least one molecule of mannose, teichoic acid, zymosan, the polysaccharide capsule of cryptococcus neoformans serotype C, Protamine, heparinase, cobra venom factor in a form adapted to enhance production of C3b, cobra venom factor in the form of dCVF, Nickel in a form adapted to enhance C3 convertase activity, sulfated polyanions, heat shock proteins, Type III repeat extra domain A of fibronectin, low-molecular weight oligosaccharides of hyaluronic acid, polysaccharide fragments of heparin sulfate, fibrinogen, lipopolysaccharides, phosphorylcholine, uric acid, IgGI and IgGIII antibodies, complement proteins and combinations thereof.

10. The method of claim 1, wherein said method further comprises the steps of preparing a viral vaccine from said collected at least one conditionally live virion and adding a pharmaceutically acceptable carrier and at least one pharmaceutically acceptable adjuvant.

11. The method of claim 1, wherein said at least one viral DNA or RNA molecule representing a complete viral genome is isolated from HIV infected tissue of an intact host.

12. The method of claim 1, wherein said at least one viral DNA or RNA molecule representing a complete viral genome is selected from a group consisting of HIV infected seminal, vaginal, and rectal tissue and is isolated from an intact host.

13. The method of claim 1, wherein said at least one viral DNA or RNA molecule representing a complete viral genome is selected from a group consisting of HIV infected seminal, vaginal, and rectal fluid and is isolated from an intact host.

14. The method of claim 1, wherein said at least one replication protein gene or corresponding mRNA is selected from the sequences consisting of those corresponding to proteins Vif, Vpr, Vpu, Tat exon 1, Vpx, and combinations thereof.

15. An immunogenic composition comprising:
a. a viral DNA or RNA representing a viral genome in which at least one replication protein gene or corresponding mRNA has been modified to render the viral DNA or RNA replication incompetent; and
b. wherein the viral DNA or RNA is encapsulated by viral proteins self-assembled in a cell-free expression system forming a conditionally live virion.

16. The immunogenic composition as claimed in claim 15, wherein said conditionally live virion is deficient in replication ability relative to a corresponding virion lacking a modification to its viral DNA or RNA.

17. The immunogenic composition as claimed in claim 15, wherein said viral genome is an HIV virus genome.

18. An immunogenic composition as claimed in claim 15, wherein said immunogenic composition is formulated as a vaccine.

19. An immunogenic composition as claimed in claim 15, wherein said immunogenic composition is formulated as a vaccine in combination with a predetermined quantity of exogenous replication protein(s) corresponding to said at least one modified replication protein gene or corresponding mRNA, thereby enabling limited replication of said conditionally live virion upon administration into a system with conditions suitable for replication.

20. An immunogenic composition as claimed in claim 15, wherein said immunogenic composition is formulated as a vaccine in combination with a predetermined quantity of exogenous replication protein(s) corresponding to said at least one modified replication protein gene or corresponding mRNA, wherein the exogenous replication protein(s) is a biologically active fragment or derivative of said modified replication protein gene or corresponding mRNA thereby enabling limited replication of the conditionally live virion upon administration into a system with conditions suitable for replication.

21. The immunogenic composition as claimed in claim 15 in combination with a pharmaceutically acceptable carrier.

22. The immunogenic composition as claimed in claim 15 in combination with at least one pharmaceutically acceptable adjuvant.

23. The immunogenic composition as claimed in claim 15 in combination with polysaccharides composed of at least one molecule of mannose.

24. The immunogenic composition as claimed in claim 15 in combination with teichoic acid.

25. The immunogenic composition as claimed in claim 15 in combination with zymosan.

26. The immunogenic composition as claimed in claim 15 in combination with the polysaccharide capsule of cryptococcus neoformans serotype C.

27. The immunogenic composition as claimed in claim 15 in combination with Protamine.

28. The immunogenic composition as claimed in claim 15 in combination with heparinase.

29. The immunogenic composition as claimed in claim 15 in combination with cobra venom factor in a form adapted to enhance production of C3.

30. The immunogenic composition as claimed in claim 15 in combination with cobra venom factor in the form of dCVF.

31. The immunogenic composition as claimed in claim 15 in combination with Nickel in a form adapted to enhance C3 convertase activity.

32. The immunogenic composition as claimed in claim 15 in combination with sulfated polyanions.

33. The immunogenic composition as claimed in claim 15 in combination with heat shock proteins.

34. The immunogenic composition as claimed in claim 15 in combination with Type III repeat extra domain A of fibronectin.

35. The immunogenic composition as claimed in claim 15 in combination with low-molecular weight oligosaccharides of hyaluronic acid.

36. The immunogenic composition as claimed in claim 15 in combination with polysaccharide fragments of heparin sulfate.

37. The immunogenic composition as claimed in claim 15 in combination with fibrinogen.

38. The immunogenic composition as claimed in claim 15 in combination with lipopolysaccharides.

39. The immunogenic composition as claimed in claim 15 in combination with phosphorylcholine.

40. The immunogenic composition as claimed in claim 15 in combination with uric acid.

41. The immunogenic composition as claimed in claim 15 in combination with IgGI and IgGIII antibodies.

42. The immunogenic composition as claimed in claim 15 in combination with complement proteins.

43. The immunogenic composition as claimed in claim 15 in combination with a pharmaceutically acceptable carrier and at least one pharmaceutically acceptable adjuvant.

44. The immunogenic composition as claimed in claim 15, wherein said viral DNA or RNA molecule representing a viral genome is isolated from HIV infected tissue.

45. The immunogenic composition as claimed in claim 15, wherein said at least one viral DNA or RNA molecule representing a viral genome is selected from a group consisting of HIV infected seminal, vaginal and rectal tissue and isolated from an intact host.

46. The immunogenic composition as claimed in claim 15, wherein said at least one viral DNA or RNA molecule representing a viral genome is selected from a group consisting of HIV infected seminal, vaginal and rectal fluid and isolated from an intact host.

47. The immunogenic composition as claimed in claim 15, wherein said at least one replication protein gene or corresponding mRNA modified is selected from the sequences consisting of those corresponding to proteins Vif, Vpr, Vpu, Tat exon 1, Vpx, and combinations thereof.

48. The immunogenic composition as claimed in claim 15, wherein said immunogenic composition is administered, orally, transbucally, transmucosally, sublingually, nasally, rectally, vaginally, intraocularly, intramuscularly, intralymphatically, intravenously, subcutaneously, transdermally, intradermally, intra tumor, topically, transpulmonarily, by inhalation, by injection, or by implantation.

49. A method for the production of a viral vaccine, comprising the steps of:
a. culturing a cell in the presence of at least one HIV virion, said culturing being under conditions suitable for viral replication, said virion having a modification in at least one replication protein gene to form a conditionally live virion;
b. adding exogenous replication protein(s) corresponding to said at least one replication protein gene or corresponding mRNA having said modification to facilitate replication of said virion in said culture in order to produce a pharmaceutically acceptable quantity of replication incompetent virions;
c. purifying and collecting said replication incompetent virions; and
d. formulating said replication incompetent virions with a predetermined quantity of replication protein(s) corresponding to said at least one replication protein gene having the modification, thereby enabling limited replication of said conditionally live virion upon administration into a system with conditions suitable for replication.

50. The method of claim 49, wherein said conditionally live virion is deficient in replication ability relative to a corresponding virion lacking a modification to its viral DNA or RNA.

51. The method of claim 49, wherein said predetermined quantity of exogenous replication protein(s) corresponding to said modified replication protein gene or corresponding mRNA, is a biologically active fragment or derivative of said modified replication protein gene or corresponding mRNA thereby enabling limited replication of the at least one conditionally live virion.

52. The method of claim 49, wherein said viral vaccine is formulated in combination with a pharmaceutically acceptable carrier.

53. The method of claim 49, wherein said viral vaccine is formulated in combination with at least one pharmaceutically acceptable adjuvant.

54. The method of claim 49, wherein said viral vaccine is formulated in combination with at least one pharmaceutically acceptable adjuvant selected from the group consisting of, polysaccharides composed of at least one molecule of mannose, teichoic acid, zymosan, the polysaccharide capsule of cryptococcus neoformans serotype C, Protamine, heparinase, cobra venom factor in a form adapted to enhance production of C3b, cobra venom factor in the form of dCVF, Nickel in a form adapted to enhance C3 convertase activity, sulfated polyanions, heat shock proteins, Type III repeat extra domain A of fibronectin, low-molecular weight oligosaccharides of hyaluronic acid, polysaccharide fragments of heparin sulfate, fibrinogen, lipopolysaccharides, phosphorylcholine, uric acid, IgGI and IgGIII antibodies, complement proteins and combinations thereof.

55. The method of claim 49, wherein said viral vaccine is formulated in combination with a pharmaceutically acceptable carrier and at least one pharmaceutically acceptable adjuvant.

56. The method of claim 49, wherein said at least one HIV virion is isolated from HIV infected tissue of an intact host.

57. The method of claim 49, wherein said at least one HIV virion is selected from a group consisting of HIV infected seminal, vaginal, and rectal tissue and is isolated from an intact host.

58. The method of claim 49, wherein said at least one HIV virion is selected from a group consisting of HIV infected seminal, vaginal, and rectal fluid and is isolated from an intact host.

59. The method of claim 49, wherein the at least one replication protein gene or corresponding mRNA is selected from the sequences consisting of those corresponding to proteins Vif, Vpr, Vpu, Tat exon 1, Vpx, and combinations thereof.

60. An immunogenic composition comprising:
a. an HIV virion having a modification in at least one replication protein gene or corresponding mRNA, forming a conditionally live virion, cultured under conditions suitable for viral replication that include exogenously added protein(s) wherein said protein(s) corresponds to said modification in at least one replication protein gene or corresponding mRNA; and
b. biologically active protein(s) corresponding to said at least one replication protein gene or corresponding mRNA having a modification, wherein said biologically active protein(s) is selected from the group consisting of whole proteins, protein fragments, protein derivatives, and combinations thereof.

61. The immunogenic composition of claim 60, wherein said conditionally live virion is deficient in replication relative to a corresponding unmodified HIV virion.

62. The immunogenic composition of claim 60, wherein said HIV virion is isolated from HIV infected tissue.

63. The immunogenic composition of claim 60, wherein said HIV virion is selected from a group consisting of HIV infected seminal, vaginal and rectal tissue and is isolated from an intact host.

64. The immunogenic composition of claim 60, wherein said HIV virion is selected from a group consisting of HIV infected seminal, vaginal, and rectal fluid and is isolated from an intact host.

65. The immunogenic composition of claim 60, wherein said at least one replication protein gene or corresponding mRNA modified is selected from the sequences consisting of those corresponding to proteins Vif, Vpr, Vpu, Tat exon 1, Vpx, and combinations thereof.

66. An immunogenic composition as claimed in claim 60, wherein said immunogenic composition is formulated as a vaccine.

67. The immunogenic composition as claimed in claim 60 in combination with a pharmaceutically acceptable carrier.

68. The immunogenic composition as claimed in claim 60 in combination with at least one pharmaceutically acceptable adjuvant.

69. The immunogenic composition as claimed in claim 60 in combination with polysaccharides composed of at least one molecule of mannose.

70. The immunogenic composition as claimed in claim 60 in combination with teichoic acid.

71. The immunogenic composition as claimed in claim 60 in combination with zymosan.

72. The immunogenic composition as claimed in claim 60 in combination with the polysaccharide capsule of cryptococcus neoformans serotype C.

73. The immunogenic composition as claimed in claim 60 in combination with Protamine.

74. The immunogenic composition as claimed in claim 60 in combination with heparinase.

75. The immunogenic composition as claimed in claim 60 in combination with cobra venom factor in a form adapted to enhance production of C3.

76. The immunogenic composition as claimed in claim 60 in combination with cobra venom factor in the form of dCVF.

77. The immunogenic composition as claimed in claim 60 in combination with Nickel in a form adapted to enhance C3 convertase activity.

78. The immunogenic composition as claimed in claim 60 in combination with sulfated polyanions.

79. The immunogenic composition as claimed in claim 60 in combination with heat shock proteins.

80. The immunogenic composition as claimed in claim 60 in combination with Type III repeat extra domain A of fibronectin.

81. The immunogenic composition as claimed in claim 60 in combination with low-molecular weight oligosaccharides of hyaluronic acid.

82. The immunogenic composition as claimed in claim 60 in combination with polysaccharide fragments of heparin sulfate.

83. The immunogenic composition as claimed in claim 60 in combination with fibrinogen.

84. The immunogenic composition as claimed in claim 60 in combination with lipopolysaccharides.

85. The immunogenic composition as claimed in claim 60 in combination with phosphorylcholine.

86. The immunogenic composition as claimed in claim 60 in combination with uric acid.

87. The immunogenic composition as claimed in claim 60 in combination with IgGI and IgGIII antibodies.

88. The immunogenic composition as claimed in claim 60 in combination with complement proteins.

89. The immunogenic composition as claimed in claim 60 in combination with a pharmaceutically acceptable carrier and at least one pharmaceutically acceptable adjuvant.

90. The immunogenic composition as claimed in claim 60, wherein said immunogenic composition is administered, orally, transbucally, transmucosally, sublingually, nasally, rectally, vaginally, intraocularly, intramuscularly, intralymphatically, intravenously, subcutaneously, transdermally, intradermally, intra tumor, topically, transpulmonarily, by inhalation, by injection, or by implantation.
